(19) United States
(12) Patent Application Publication
(10) Pub. No.: US 2023/0284683 A1
(43) Pub. Date: Sep. 14, 2023

(54) VAPORIZER FOR CONTROLLED AEROSOLIZATION OF CANNABINOID CONCENTRATES

(71) Applicant: ROXX LABS LTD., Migdal Haemeq (IL)

(72) Inventor: Miron HAZANI, Haifa (IL)

(21) Appl. No.: 18/019,523

(22) PCT Filed: Aug. 4, 2021
(86) PCT No.: PCT/IL2021/050945

§ 371 Date: Feb. 3, 2023

Related U.S. Application Data
(60) Provisional application No. 63/134,638, filed on Jan. 7, 2021.
(60) Provisional application No. 63/073,950, filed on Sep. 3, 2020.
(60) Provisional application No. 63/061,847, filed on Aug. 6, 2020.

(51) Int. Cl. A24F 40/30; A24F 40/42; A24F 40/465

(52) U.S. Cl. CPC A24F 40/30; A24F 40/42; A24F 40/465

(57) ABSTRACT

The present disclosure generally relates to the field of aerosol generation devices, and more particularly to vaporizers configured to generation of aerosols from cannabinoid concentrates in a dose controlled manner.

### FIELD OF THE INVENTION

The present disclosure generally relates to the field of aerosol generation devices, and more particularly vaporizers configured for generation of aerosols from cannabinoid concentrates in a dose controlled manner.

### BACKGROUND OF THE INVENTION

Electronic devices, such as vaporizers and cigarettes typically function as condensation aerosol generators, which operate by vaporizing a liquid such as a nicotine-based composition or cannabis-based composition via heat applied by a heat source. Upon cooling, the vapor condenses to form an aerosol comprising droplets of liquid or particles which can be inhaled by a user through a mouthpiece.

Most aerosol generating devices designed for consumption of cannabis products are conventionally referred as vaporizers and/or vaping devices. The heated composition in such known devices is liquid. Such compositions usually include solutions, suspensions or emulsions containing cannabinoids.

The liquid compositions of cannabinoids are usually a mixture of cannabis product with humectants, having relatively low latent heat of vaporization, such as propylene glycol (PG) or vegetable glycerin (VG). The liquid mixture is typically drawn into a wicking material that is in contact with a heating element, which may consist a coil of a conducting material to be heated when electric current is driven there through. When not contacted with a liquid, or after the liquid is substantially evaporated the temperature of the coil can reach in some instances a value of over 800 degrees Celsius.

One particular drawback stems from the fact that such products, while carrying a smaller risk than that associated with conventional vaporizers, still present health risks due to the evolution of hazardous compounds arising from heating propylene glycol and vegetable glycerin to elevated temperatures, as well as pyrolysis products of over-heated cannabinoids.

A cannabis concentrate (also called marijuana concentrate, marijuana extract, or cannabis extract) is a highly potent tetrahydrocannabinol (THC) and/or cannabidiol (CBD) concentrated mass. Cannabis concentrates contain high THC levels that may range from 40 to 80%, up to four times stronger in THC content than high grade or top shelf marijuana, which normally measures around 20% THC levels.

Cannabis concentrates are available and are typically consumed directly through inhalation after being evaporated by exposure to direct flame or heating element. Specifically, cannabis concentrates are highly viscous and their users typically place the concentrate on a simple platform, such as a metallic or ceramic crucible, and use a torch or hot nail to evaporate the concentrate and inhale the cannabis vapor formed above. Such procedures are conventional among cannabis concentrates users since they provide highly concentrated THC and/or CBD dosages. However, such procedures are highly inaccurate and cannot be used to control the consumed dose.

There is an unmet need for devices, (e.g. vaporizers) capable of evaporating cannabis concentrates. Moreover, there is an unmet need for such devices, which enable dosage control of the consumed cannabinoids from the cannabis concentrate composition. For example, a cannabis concentrate user may want to consume a known amount of THC or CBD, which is in accordance with a medical prescription or with other health limitations.

### SUMMARY OF THE INVENTION

The present invention generally relates to the field of aerosol generation devices, and more particularly to vaporizers configured to generate aerosols from concentrate cannabinoid compositions, which are typically viscous by nature.

According to some embodiments, there is provided an aerosol generating device. The device is specifically intended to produce cannabinoid containing vapor from concentrated cannabinoid compositions. Specifically, the device disclosed herein is configured to aerosolize cannabinoid concentrates. More specifically, the device disclosed herein is configured to vaporized and aerosolize concentrated and viscous cannabinoid compositions.

The term “concentrate”, as used herein, refers to cannabis products made from the cannabis plant that have been processed to keep only the most desirable plant compounds (primarily the cannabinoids and sometimes terpenes), while removing excess plant material and other impurities. The natural product are usually extracted from the raw plant material using either organic solvents, such as ethanol, butane, propane and hexane, or supercritical carbon dioxide. Therefore, cannabinoid concentrates have a greater proportion of cannabinoids and terpenes when compared to natural cannabis flowers. Cannabis concentrates are sometimes also called marijuana concentrate, marijuana extract, or cannabis extract. They are highly potent tetrahydrocannabinol (THC) and/or cannabidiol (CBD) concentrated mass. THC rich concentrates typically contain high THC levels that may range from 40 to 80% THC by weight, up to four times stronger in THC content than high grade or top shelf marijuana. Distilled concentrate was reportedly available at 99.58% THC content.

Cannabinoids, such as THC and CBD are usually present as viscous oils at room temperature. As such, cannabinoid concentrates are typically highly viscous compositions. These viscous compositions are usually taken by cannabis consumers with a metal teaspoon and heated using a direct flame to produce cannabinoid vapor, which is then inhaled and consumed. Being viscous, cannabis concentrates are highly difficult to handle. The device disclosed herein, provide a solution to the handling of these viscous compositions for inhalation, through a simple to use vaporizer, which may be hand-held and operated by non-expert cannabis consumers

In addition, it is understood that the high concentration of the cannabinoids in the cannabinoid concentrates may come with two associated risks: (a) unintentional consumption of higher (or alternatively lower) amounts of cannabinoids by a user, as compared to the user desired or need. Often cannabis consumers wish to monitor the daily amount or amount per use. However, the high viscosity of cannabinoid concentrates prevents such measurements and monitoring. (b) There is a risk of abusing the commercial availability, where available, of cannabis concentrates by consumers to consume overdosed amount of cannabinoids. (c) In cases that cannabinoid are medically prescribed by a physician, the dosing control and monitoring of cannabinoid concentrates consumption is not currently possible, leading to using alternative compositions in prescriptions.

However, it is emphasized that using cannabinoid concentrates may be preferable over using other cannabis compositions (e.g. cannabis plant or dissolved cannabinoids), as the concentrates are both substantially free from organic solvents and do not include many plant material, which are burned as inhaled as smoke.

The device disclosed herein provides solution to the problems associated to cannabinoid concentrates presented above.

Specifically, according to some embodiments, the aerosol generating device disclosed herein comprises a tray comprising a plurality of wells wherein at least some of the wells contain a known measured amount of cannabinoid concentrate within their cavity. As specified below, the known amount of cannabinoid concentrate and/or of the specific cannabinoids within the concentrates is a key factor in monitoring and determining the amount of consumed cannabinoids, according to some embodiments. The aerosol generating device further comprises a plurality of heaters, each heater is configured to elevate the temperature of one respective well of the plurality of wells; and a processing unit configured to separately operate each heater, thereby to elevate the temperature within each well individually, according to some embodiments.

The individual control over individual well, each containing a known amount of cannabinoids results in the control over the dosing and the monitoring thereof.

According to some embodiments, there is provided an aerosol generating device, which comprises: a tray comprising a plurality of wells, each having an open side, a closed face and a cavity there between, wherein at least some of the wells contain a cannabinoid concentrate within their cavity; a plurality of heaters, each heater is configured to elevate the temperature of one respective well of the plurality of wells; a processing unit configured to separately operate each heater, thereby to elevate the temperature within each well individually; and an outlet. According to some embodiments, the closed face of each well is facing the processing unit and the open side of each well is facing the outlet. Specifically, since the closed face of each well is facing the processing unit and the open side of each well is facing the outlet, the vapor produced by the elevation of temperature within the wells is confined to flow in the direction from the tray to the outlet.

According to some embodiments, each heater is in contact with the closed face of the well heated thereby.

According to some embodiments, each heater resides at least partially inside the cavity of the well heated thereby.

According to some embodiments, each cavity has a volume in the range of 0.5-10 microliters. According to some embodiments, each of the plurality of well has the same volume as the other wells.

According to some embodiments, each cannabinoid concentrate contained within one of the cavities has mass in the range of 0.5 to 5 milligrams. According to some embodiments, each of the wells comprises a cannabinoid concentrate within its cavity. According to some embodiments, each well comprises the same amount of cannabinoid concentrate within its cavity as the other wells.

According to some embodiments, each heater is configured to elevate the temperature of one respective well of the plurality of wells, thereby to produce cannabinoid vapor from the cannabinoid concentrate.

According to some embodiments, the aerosol generating device comprises a mouthpiece. According to some embodiments, the mouthpiece extends between the outlet and a proximal mouthpiece side, which faces the open side of the plurality of heaters. According to some embodiments, the proximal mouthpiece side is tapering towards the outlet. According to some embodiments, upon production of the cannabinoid vapor, the vapor flows from the open side, through the proximal face and out the outlet. According to some embodiments, during said flowing the vapor at least partially condenses to produce a cannabinoid aerosol.

According to some embodiments, each cannabinoid concentrate contained within one of the cavities comprises 0.25 to 5 milligrams of a cannabinoid selected from tetrahydrocannabinol (THC), cannabidiol (CBD) or both.

According to some embodiments, the total mass of the cannabinoid concentrate within the tray cavities is in the range of 40 to 500 milligrams.

According to some embodiments, the processing unit is electrically connected to each heater through an electric driver.

According to some embodiments, each of the plurality of heaters is a coil heater.

According to some embodiments, the cannabinoid concentrate has a viscosity of at least 1000 mPa·s.

According to some embodiments, the tray comprises a thermally insulating material.

According to some embodiments, each one of the plurality of wells is thermally isolated from the other wells.

According to some embodiments, the aerosol generating device comprises:
a controlling member comprising the processing unit, a power source compartment and a housing, wherein the housing houses the processing unit and the power source compartment; and
a cartridge comprising the tray, the plurality of heaters and the outlet, wherein the outlet is constructed as part of a mouthpiece.

According to some embodiments, the controlling member is connectable to the cartridge. According to some embodiments, upon connection the aerosol generating device is assembled. According to some embodiments, upon assembly, the processing unit forms an electric contact with each one of the heaters separately.

According to some embodiments, the housing comprises a proximal face and the cartridge comprises a distal face. According to some embodiments, upon assembly the housing proximal face is facing the cartridge distal face. According to some embodiments, the controlling member comprises a reader adjacent to the housing proximal face and the cartridge comprises an identifier at an external surface of its distal face. According to some embodiments, said identifier is positioned to face the reader upon assembly. According to some embodiments, the reader is configured to identify the identifier and to send identification signals indicative of the identification to the processing unit.

According to some embodiments, the identification comprises information about contents within each cavity of the plurality of wells.

According to some embodiments, the identifier is a barcode and the reader is a barcode reader.

According to some embodiments, the aerosol generating device comprises:
a housing, which houses the processing unit, a mouthpiece comprising the outlet and a power source compartment; and
a cassette comprising the tray and the plurality of heaters.

According to some embodiments, the housing comprises an inlet slot configured for insertion of the cassette into the housing. According to some embodiments, upon insertion of cassette into the inlet slot, the aerosol generating device is assembled. According to some embodiments, upon assembly, the processing unit forms an electric contact with each one of the heaters separately.

According to some embodiments, the aerosol generating device further comprises a reader housed within the housing. According to some embodiments, the cassette comprises an identifier at an external surface of distal face thereof. According to some embodiments, upon assembly said identifier is positioned to face the reader. According to some embodiments, the reader is configured to identify the identifier and to send identification signals indicative of the identification to the processing unit. According to some embodiments, the identification comprises information about contents within each cavity of the plurality of wells. According to some embodiments, the identifier is a barcode and the reader is a barcode reader.

According to some embodiments, wells containing a cannabinoid concentrate within their cavity are produced by insertion of the cannabinoid concentrate into the well cavities. According to some embodiments, the insertion is performed by a procedure selected from:
placing undissolved cannabinoid concentrates over the tray and depositing the concentrates into the cavities used a doctor blade; and
depositing dissolved cannabinoid concentrates in the cavities and evaporating the solvent, optionally a plurality of times.

According to some embodiments, the insertion is performed by placing undissolved cannabinoid concentrates over the tray and depositing the concentrates into the cavities used a doctor blade. According to some embodiments, the insertion is performed by depositing dissolved cannabinoid concentrates in the cavities and evaporating the solvent, optionally a plurality of times. According to some embodiments, the insertion is performed by depositing dissolved cannabinoid concentrates in the cavities and evaporating the solvent a plurality of times.

According to some embodiments, each heater has a total resistance in the range of 0.2 to 2 Ohms.

According to some embodiments, each heater is configured to provide an energy output in the range of 1 to 50 Watts.

According to some embodiments, each heater is configured to elevate the temperature of a well heated thereby to a predetermined temperature. According to some embodiments, the processing unit is configured to separately operate each heater, thereby to elevate the temperature within each well individually to the predetermined temperature. According to some embodiments, upon being heated to the predetermined temperature, the cannabinoid concentrate is being evaporated to form cannabinoid vapor at a predetermined rate.

According to some embodiments, the predetermined temperature is in the range of 160° C. to 480° C. According to some embodiments, the predetermined rate is in the range of 1 to 1000 micrograms per second.

According to some embodiments, the processing unit is configured to simultaneously operate n of the plurality of heaters, wherein n is an integer greater than 1. According to some embodiments, each of then heaters is configured to elevate the temperature of a well containing a cannabinoid concentrate within its cavity. According to some embodiments, upon the simultaneous operation of the n heaters a cannabinoid vapor is formed at a rate substantially equal to n times the predetermined rate.

According to some embodiments, n of the plurality of heaters are each configured to elevate the temperature of a well containing a cannabinoid concentrate within its cavity and m of the plurality of heaters are each configured to elevate the temperature of a well containing a second composition within its cavity. According to some embodiments, each of n and m is a an integer greater than zero. According to some embodiments, the processing unit is configured to simultaneously operate each of the n and m of the plurality of heaters. According to some embodiments, upon the simultaneous operation of the n and m of the plurality of heaters a cannabinoid vapor is formed at a rate substantially equal to n times the predetermined rate and a vapor of the second composition is formed at a rate substantially equal to m times a second predetermined rate.

According to some embodiments, the predetermined rate is at least a half and not more than twice the second predetermined rate.

According to some embodiments, n is not equal to m.

According to some embodiments, the second composition has a viscosity of at least 1000 mPa·s.

According to some embodiments, the second composition is a second cannabinoid concentrate.

According to some embodiments, the processing unit is configured to separately operate each heater to controllably elevate the temperature within the well heated thereby to a controlled temperature. According to some embodiments, upon controlling said controlled temperature, the cannabinoid concentrate is being evaporated, to form cannabinoid vapor at a controlled rate. According to some embodiments, at least some of the wells contain a second composition within their cavity, wherein the processing unit is configured to control the temperature of each of the wells containing the cannabinoid concentrate and to control the temperature of each of the wells containing the second composition, thereby to control both the rate of the cannabinoid vapor and a rate of the formation of vapor of the second composition. According to some embodiments, the second composition has a viscosity of at least 1000 mPa·s. According to some embodiments, wherein the controlled temperature is in the range of 160° C. to 480° C. and the controlled rate is in the range of 1 to 1000 micrograms per second.

According to some embodiments, there is provided an aerosol generating system comprising the aerosol generating device disclosed herein and a user interface configured to send instruction signals to the processing unit.

According to some embodiments, the user interface is embedded on the aerosol generating device. According to some embodiments, the user interface is electrically wired to the processing unit and is configured to send electric signals thereto. According to some embodiments, the processing unit is configured to send electric signals to the user interface.

According to some embodiments, the user interface is embedded on an external device, wherein the user interface comprises a transmitter and the processing unit comprises a receiver. According to some embodiments, the user interface is configured to send wireless signals to the processing unit through its transmitter, to be received by the processing unit receiver.

According to some embodiments, the processing unit comprises a transmitter and the user interface comprises a receiver, wherein the processing unit is configured to send wireless signals to the user interface through its transmitter, to be received by the user interface receiver.

According to some embodiments, the user interface is configured to send instruction signals to the processing unit to effect at least one parameter selected from the group consisting of: n, the predetermined rate and the predetermined temperature. Each possibility represents a separate embodiment. According to some embodiments, the parameter is n. According to some embodiments, the parameter is the predetermined rate. According to some embodiments, the parameter is the predetermined temperature.

According to some embodiments, the instruction signals effect at least one parameter selected from the group consisting of: n and the predetermined temperature, thereby effecting the predetermined rate. Each possibility represents a separate embodiment.

According to some embodiments, the user interface is configured to send instruction signals to the processing unit to effect the predetermined rate. According to some embodiments, the user interface is further configured to send instruction signals to the processing unit to effect a duration of the operation of the heaters, thereby controlling the total amount of cannabinoid concentrate being evaporated.

According to some embodiments, the user interface is controllable by a user, and at least one of the parameters is controllable by the user.

According to some embodiments, the user interface requires a permit, and control of the total amount of cannabinoid concentrate being evaporated is controllable by a holder of the permit. According to some embodiments, the holder of the permit is a physician.

According to some embodiments, wherein the user interface is further configured to send instruction signals to the processing unit to effect at least one parameter selected from the group consisting of: m and the second predetermined rate.

According to some embodiments, the processing unit is configured to calculate the amount of cannabinoid concentrate evaporated and to record results of said calculation, to send wireless recordation signals to the user interface, wherein the wireless recordation signals are indicative of said recording.

According to some embodiments, the calculation is based on at least one parameter selected from the group consisting of n, the predetermined rate and the predetermined temperature.

According to some independent embodiments, an aerosol generating device is provided, the aerosol generating device comprising: a rotatable tray comprising at least one well having an open side, a closed face and a cavity there between, wherein the at least one well contains a cannabinoid concentrate within its cavity; a rotatable tray actuator configured to rotate the rotatable tray around a rotational axis; at least one heater juxtaposed with the rotatable tray; a processing unit configured to operate the rotatable tray actuator and the at least one heater; and an outlet, wherein the open side of the at least one well faces the outlet.

According to some embodiments, the at least one heater is configured to elevate the temperature of the at least one well.

According to some embodiments, the at least one well comprises a plurality of wells, the plurality of wells radially arrayed about the rotational axis.

According to some embodiments, each of the plurality of wells is thermally isolated from the other wells.

According to some embodiments, the plurality of wells comprises a first set of wells and a second set of wells, and wherein the first set of wells are radially arrayed about the rotational axis and the second set of wells are radially arrayed about the first set of wells.

According to some embodiments, each of the first set of wells contains a first type of cannabinoid concentrate within its cavity, wherein each of the second set of wells contains a second type of cannabinoid concentrate within its cavity.

According to some embodiments, the at least one heater comprises a pair of heaters, a first of the pair of heaters juxtaposed with the first set of wells and the second pair of heaters juxtaposed with the second set of wells such that a distance between the rotational axis and the second heater is greater than a distance between the rotational axis and the first heater.

According to some embodiments, the at least one well exhibits a shape radially extending about the rotational axis.

According to some embodiments, the at least one well comprises a pair of wells, each of the pair of wells exhibiting a respective shape radially extending about the rotational axis, wherein a first of the pair of wells extends radially about the rotation axis and the second of the pair of wells extends radially about the first of the pair of wells.

According to some embodiments, the first of the pair of wells contains a first type of cannabinoid concentrate within its cavity, wherein the second of the pair of wells contains a second type of cannabinoid concentrate within its cavity.

According to some embodiments, the at least one heater comprises a pair of heaters, a first of the pair of heaters juxtaposed with the first of the pair of wells and the second pair of heaters juxtaposed with the second of the pair of wells such that a distance between the rotational axis and the second heater is greater than a distance between the rotational axis and the first heater.

According to some embodiments, the rotatable tray actuator comprises a gear comprising a plurality of teeth, wherein the rotatable tray comprises a plurality of teeth, the plurality of teeth of the rotatable tray configured to mesh with the plurality of teeth of the gear.

According to some embodiments, the rotatable tray actuator comprises an axle secured to the rotatable tray and extending along the rotational axis.

According to some embodiments, the aerosol generating device further comprises at least one translation mechanism configured to translate the at least one heater between a first position and a second position in relation to the rotatable tray, a distance between the first position and the rotatable tray being less than a distance between the second position and the rotatable tray, wherein the processing unit is further configured to: operate the at least one translation mechanism to translate the at least one heater from the first position to the second position; operate the rotatable tray actuator to rotate the rotatable tray about the rotation axis by a predetermined amount; and subsequent to the rotation of the rotatable tray, operate the at least one translation mechanism to translate the at least one heater from the second position to the first position.

According to some embodiments, in the first position the at least one heater is in contact with the rotatable tray.

According to some embodiments, each of the at least one well has a volume in the range of 0.5-10 microliters.

According to some embodiments, the aerosol generating device comprises: a housing; a cassette positioned within the housing; an identifier positioned on a face of the cassette; and a reader positioned within the housing, wherein the rotatable tray is positioned within the cassette, wherein the at least one heater, the rotatable tray actuator and the processing unit are positioned within the housing, external to the cassette, and wherein the reader is configured to: identify the identifier; and output a signal indicative of an identification of the identifier.

According to some embodiments, the aerosol generating device comprises: a cassette, the rotatable tray positioned within the cassette; and a housing, wherein the cassette is detachably attachable within the housing, and wherein the at least one heater, the rotatable tray actuator and the processing unit are positioned within the housing, external to the cassette.

According to some embodiments, the housing comprises an inlet slot configured and dimensioned to allow the cassette to be inserted therethrough, the cassette juxtaposed with the inlet slot when detachably attachable within the housing.

According to some embodiments, the housing comprises a mouthpiece extending from the outlet, wherein the mouthpiece is hingeably or detachably attachable to the housing.

According to some embodiments, the aerosol generating device comprises: a housing; a cartridge detachably coupled to the housing; an identifier positioned on the cartridge; and a reader secured to the housing, wherein the rotatable tray is positioned within the cartridge, wherein the at least one heater, the rotatable tray actuator and the processing unit are positioned within the housing, external to the cartridge, wherein the housing comprises a mouthpiece extending from the outlet, and wherein the reader is configured to: identify the identifier; and output a signal indicative of an identification of the identifier.

According to some embodiments, the at least one heater comprises at least one induction coil.

According to some embodiments, the at least one heater comprises at least one laser.

Other objects, features and advantages of the present invention will become clear from the following description, examples and drawings.

### BRIEF DESCRIPTION OF THE DRAWINGS

FIG. 1 constitutes a cross sectional view of an assembled aerosol generating device comprising a cartridge and a controlling member, when connected, according to some embodiments.

FIG. 2 constitutes a cross sectional view of a disassembled aerosol generating device comprising a cartridge and a controlling member, when disconnected, according to some embodiments.

FIG. 3 constitutes a cross sectional view of an assembled aerosol generating device comprising a cartridge and a controlling member, when connected, wherein the cartridge comprises partially filled wells, according to some embodiments.

FIG. 4 constitutes a cross sectional view of an assembled aerosol generating device comprising a cartridge and a controlling member, when connected, wherein the cartridge comprises partially filled wells of two different compositions, according to some embodiments.

FIG. 5 constitutes a cross sectional view of an assembled aerosol generating device comprising a cassette and a controlling member, when the cassette is inserted into the controlling member, according to some embodiments.

FIGS. 6A-B constitute cross sectional views of a cassette (FIG. 6A) and a controlling member (FIG. 6B) of a disassembled aerosol generating device, according to some embodiments.

FIG. 7 constitutes a cross sectional view of an assembled aerosol generating device comprising a cassette and a controlling member, when the cassette is inserted into the controlling member, wherein the cassette comprises partially filled wells of a cannabinoid composition, according to some embodiments.

FIGS. 8A-B constitute cross sectional views of an assembled aerosol generating device comprising a cartridge and a controlling member, when assembled, wherein the cartridge comprises filled (FIG. 8A) or partially filled (FIG. 8B) wells of two different compositions, according to some embodiments.

FIGS. 9A-C constitute a top view (FIG. 9A), cross sectional top view (FIG. 9B) and a bottom view (FIG. 9C) of a tray of an aerosol generating device, according to some embodiments. The tray comprises a plurality of wells, wherein each well is filled with a cannabinoid concentrate, according to some embodiments.

FIGS. 10A-C constitute a top view (FIG. 10A), cross sectional top view (FIG. 10B) and a bottom view (FIG. 10C) of a tray of an aerosol generating device, according to some embodiments. The tray comprises a plurality of wells, wherein some of the wells are filled with a cannabinoid concentrate and the other wells are empty, according to some embodiments.

FIGS. 11A-C constitute a top view (FIG. 11A), cross sectional top view (FIG. 11B) and a bottom view (FIG. 11C) of a tray of an aerosol generating device, according to some embodiments. The tray comprises a plurality of wells, wherein some of the wells are filled with a cannabinoid concentrate, some are filled with a second composition, and the remaining wells are empty, according to some embodiments.

FIGS. 12A-C constitute a top view (FIG. 12A), cross sectional top view (FIG. 12B) and a bottom view (FIG. 12C) of a tray of an aerosol generating device, according to some embodiments. The tray comprises a plurality of wells, wherein some of the wells are filled with a cannabinoid concentrate, some wells are filled with a second composition, some wells are partially filled with the cannabinoid concentrate, some wells are partially filled with the second composition, and the remaining wells are empty, according to some embodiments.

FIGS. 13A-13E constitute a cross-sectional view (FIG. 13A), a top view (FIG. 13B), perspective views (FIGS. 13C-13D) and an additional top view (FIG. 13E) of various portions of an aerosol generating device, according to some embodiments.

FIGS. 14A-14D constitute a top view (FIG. 14A), a perspective view (FIG. 14B), an additional top view (FIG. 14C) and an additional perspective view (FIG. 14D) of an aerosol generating device, according to some embodiments.

FIG. 15A constitutes a cross-sectional view of a portion of an aerosol generating device, according to some embodiments.

FIGS. 15B-15C constitute various conceptual illustrations of positions of one or more heaters, according to some embodiments.

FIG. 16A constitutes a cross-sectional view of an aerosol generating device in a closed configuration, according to some embodiments.

FIGS. 16B-16C constitute a top view (FIG. 16B) and a side view (FIG. 16C) of a portion of aerosol generating device of FIG. 16A, according to some embodiments.

FIG. 16D constitutes a cross-sectional view of the aerosol generating device of FIG. 16A in an open configuration, according to some embodiments.

### DETAILED DESCRIPTION

Provided herein are aerosol generating devices particularly designed for producing cannabinoid aerosols for inhalation from cannabinoid concentrates. The aerosol generating devices disclosed herein advantageously enable dosing of the consumed amounts of cannabinoids. The devices may preferably also enable monitoring of the consumed amounts of cannabinoid by a user, a care giver or a physician, according to some embodiments.

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure. In the figures, like reference numerals refer to like parts throughout. Throughout the figures of the drawings, different superscripts for the same reference numerals are used to denote different embodiments of the same elements. Embodiments of the disclosed devices and systems may include any combination of different embodiments of the same elements. Specifically, any reference to an element without a superscript may refer to any alternative embodiment of the same element denoted with a superscript. Components having the same reference number followed by different lowercase letters may be collectively referred to by the reference number alone. If a particular set of components is being discussed, a reference number without a following lowercase letter may be used to refer to the corresponding component in the set being discussed.

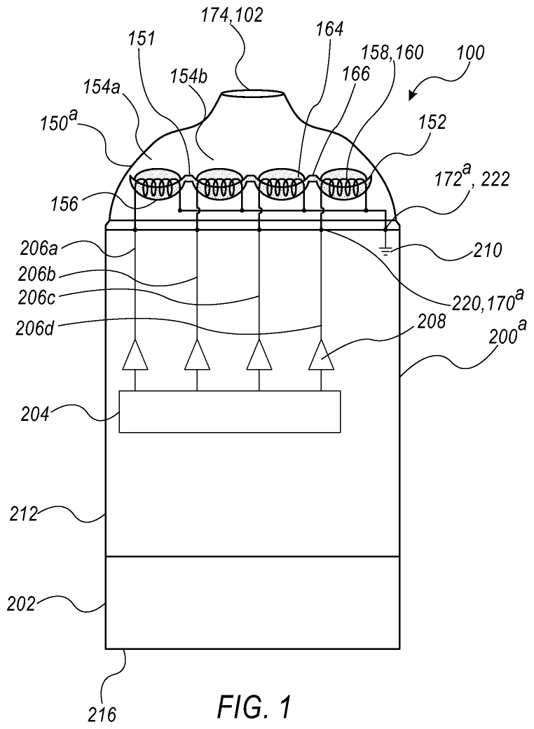

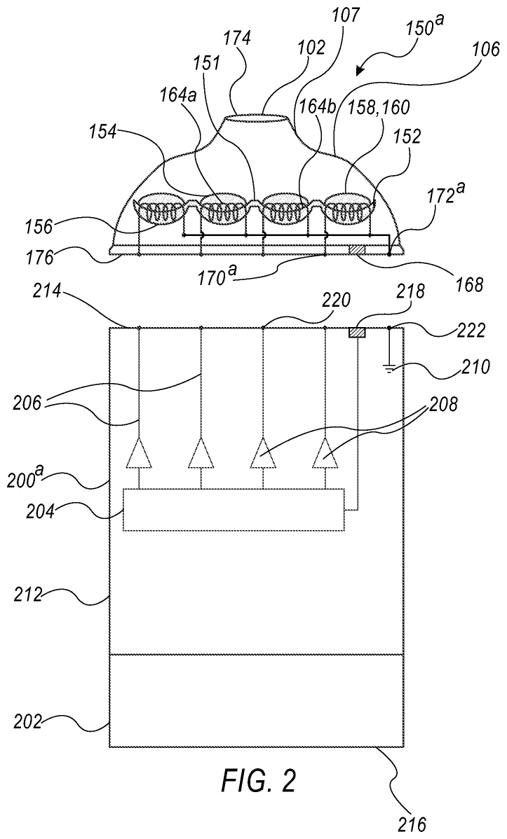

Reference is now made to FIG. 1 and FIG. 2. FIG. 1 and FIG. 2 each constitutes a schematic illustration in cross section of an aerosol generating device 100 comprising a cartridge 150^{a} and a controlling member 200^{a}, according to some embodiments.

According to some embodiments, there is provided an aerosol generating device 100. which comprises: a tray 151 comprising a plurality of wells 152, wherein at least some of the wells contain a cannabinoid concentrate 160; a plurality of heaters 164, a processing unit 204; and an outlet 102.

The term “aerosol generating device” refer to a device configured to produce a vapor or aerosol from a liquid or solid composition. aerosol generating devices are typically used to deliver a solid or liquid (including semi liquid) composition to a subject in need thereof in a inhalable form (i.e. in a substantially gaseous form). Aerosol generating devices include nebulizers and inhalers, which typically produce aerosols by application of mechanical force on the compositions (e.g. by gas flow or vacuum), and to vaporizers and electronic cigarettes, which typically heating unit(s) and produce aerosols by vaporizing the composition. In both instances, the composition is delivered through an outlet, wherein in the latter instances (i.e. vaporizers and electronic cigarettes) the vapor is usually at least partially being condensed to form droplets of the composition, through the delivery.

The present aerosol generating devices include heating units, and are typically conventionally referred to as vaporizers. Specifically, the typical convention is that aerosol generating devices for aerosolizing nicotine/tobacco compositions are called electronic cigarettes, whereas devices for aerosolizing cannabinoid/cannabis compositions are called vaporizer or vaping devices.

According to some embodiments, the aerosol produced by aerosol generating device 100 include cannabinoid particles. As used herein the terms “aerosol”, “aerosolized composition” or “aerosolized cannabinoid(s)” refer to a dispersion of solid or liquid particles in a gas. As used herein “aerosol”, “aerosolized composition” or “aerosolized cannabinoid(s)” may be used generally to refer to a material that has been vaporized, nebulized, being in a form of spray or jet or otherwise converted from a solid or liquid form to an inhalable form including suspended solid or liquid drug particles.

The term “vapor” as used herein refers to a gaseous state of matter.

As used herein, the terms “vaporization” and “evaporation” are interchangeable.

According to some embodiments, there is provided an aerosol generating device 100. According to some embodiments, aerosol generating device 100 comprises a tray 151 comprising a plurality of wells 152. According to some embodiments, each one of plurality of wells 152 has an open side 154, a closed face 156 and a well cavity 158 there between.

It is to be understood that each one of plurality of wells 152 is a 3-dimensional body, which defines a well cavity 158. This 3D body may be, e.g. an open polyhedron or an open curved polyhedron, however, since it includes an open side 154, it is not a closed structure. Being a 3D body, each well 152 has different sides, at least some of which as either contacting or opposing other side(s). Some sides are faces, such as those of closed polyhedra or curved polyhedra, whereas some sides are at least partially open, so there are usually not considered faces. For example, closed face 156 may be circular, square, rectangular, half spherical, etc. and may be considered to be a face according to the definitions of the present disclosure. Open side is open and generally not considered a face as it is open. According to some embodiments, open side 154 is opposite to closed face 156. It is to be understood that be using the term “opposite” it is not meant that the edge(s) of closed face 156 cannot come in contact or be adjacent to open side 154. For example, aerosol generating device 100 as depicted in FIGS. 1-2 has plurality of wells 152, each having an open half spherical structure with an open side between the edges of the half sphere. In this case the “solid” half spherical portion constitutes the closed face 156 and the circular side enclosed by the edge of half-sphere constitutes the open side 154.

According to some embodiments, at least some of plurality of wells 152 contain a cannabinoid concentrate 160 within well cavity 158. According to some embodiments, each of plurality of wells 152 contains a cannabinoid concentrate 160 within well cavity 158. As detailed below, some of the wells may contain a second composition 162, which may contain cannabinoids and/or other material for inhalation. Moreover, some of plurality of wells 152 may be consumed after inhalation(s), such that not all of plurality of wells 152 are containing compositions. Furthermore, tray 151 may be manufactured as a templated containing a specific number of wells 152 and specific total volume of well cavities 158, which is not in accordance with a specific, possibly lower-dosed, prescription. Thus, trays 151 may be required to be partially empty.

According to some embodiments, each heater is configured to elevate the temperature of one respective well of the plurality of wells. Specifically, according to some embodiments, the number of plurality of wells 152 is equal to the number of plurality of heaters 164, such that each heater 164 is associated with a single well 152.

It is to be understood that the phrase “heater 164 associated with well 152” is intended to mean that this specific heater 164 of plurality of heaters 164 is positioned and configured to elevated the temperature of this specific well 152, its specific well cavity 158 and the specific cannabinoid concentrate 160 within the specific well cavity 158. Similarly, it is to be understood that the phrase “heater 164 associated with cannabinoid concentrate 160” is intended to mean that this specific heater 164 of plurality of heaters 164 is positioned and configured to elevated the temperature of this specific cannabinoid concentrate 160 within the specific well cavity 158 of the specific well 152. Similarly, it is to be understood that the phrase “heater 164 associated with well cavity 158” is intended to mean that this specific heater 164 of plurality of heaters 164 is positioned and configured to elevated the temperature of this well cavity 158 of the specific well 152, and heat the specific cannabinoid concentrate 160 within the specific well cavity 158.

According to some embodiments, processing unit 204 is configured to separately operate each heater 164. It is to be understood that by separately operating each heater 164, processing unit 204 is configured to control and elevate the temperature within each well cavity 158 separately. Specifically, according to some embodiments, by separately operating each heater 164, processing unit 204 is configured to control and elevate the temperature within each well cavity 158 separately, thereby to separately control the rate of vaporization of each cannabinoid concentrate 160 within each well cavity 158. According to some embodiments, processing unit 204 is configured to separately operate each heater 164, thereby to elevate the temperature within each plurality of wells 152 individually.

According to some embodiments, closed face 156 of each one of plurality of wells 152 is facing the processing unit 204, when aerosol generating device 100 is assembled.

As detailed below, according to some embodiments, aerosol generating device 100 as shown in FIGS. 1 and 2 includes two detachably connectable parts: controlling member 200^{a} and cartridge 150^{a}. Upon connection of controlling member 200^{a} and cartridge 150^{a}, aerosol generating device 100 is said to be assembled, whereas upon disconnection between controlling member 200^{a} and cartridge 150^{a}, aerosol generating device 100 is said to be disassembled.

According to some embodiments, open side 154 of each one of plurality of wells 152 is facing the outlet 102.

Specifically, according to some embodiments, closed face 156 is opposite to open side 154 with well cavity 158 therebetween. According to some embodiments, closed face 156 of each one of plurality of wells 152 is facing away from outlet 102 and open side 154 of each one of plurality of wells 152 is facing the opposite side, i.e. facing outlet 102. Therefore, the vapor produced by the elevation of temperature within plurality of wells 152 and vaporization of cannabinoid concentrate 160 contained therein, is confined to flow in the direction from tray 151 to outlet 102.

According to some embodiments, the aerosol generating device comprises a controlling member controlling member 200^{a} and cartridge 150^{a}.

According to some embodiments, controlling member 200^{a} comprises processing unit 204, a power source compartment 202 and a controlling member housing 212. According to some embodiments, controlling member housing 212 houses processing unit 204 and power source compartment 202.

According to some embodiments, cartridge 150^{a} comprises tray 151, plurality of heaters 164 and outlet 102.

According to some embodiments, cartridge 150^{a} is intended to be disposable and for use until the cannabinoid concentrate 160 contained in its plurality of wells 152 is consumed, whereas controlling member 200^{a} is durable and after consumption of the cannabinoid concentrate 160 contained in a first cartridge 150^{a}, a second cartridge 150^{a} may be mounted/assembled on controlling member 200^{a} for a further sequence of aerosolizations.

According to some embodiments, cartridge 150^{a} is detachably attachable to controlling member 200^{a}. According to some embodiments, upon attaching cartridge 150^{a} to controlling member 200^{a}, aerosol generating device 100 is assembled. According to some embodiments, the assembly of aerosol generating device 100 entails a physical connection between cartridge 150^{a} and controlling member 200^{a}. According to some embodiments, upon assembling cartridge 150^{a} and controlling member 200^{a} electric contact is made between electrical components of cartridge 150^{a} and controlling member 200^{a} as detailed herein.

According to some embodiments, each one of plurality of heaters 164 is in contact with the closed face 156 of the well 152 heated thereby. Specifically, plurality of heaters 164 are depicted in FIGS. 1 and 2 as coil heaters connected to electric wires penetrating through closed faces 156. This results in a contact between heaters 164 and the wells 152 heated thereby.

According to some embodiments, each one of plurality of heaters 164 is a coil heater.

According to some embodiments, each one of plurality of heaters 164 resides at least partially inside one of plurality of wells 152. According to some embodiments, each one of plurality of heaters 164 resides at least partially inside one well 152 heated thereby. According to some embodiments, each one of plurality of heaters 164 resides at least partially inside the well cavity 158 of the well 152 heated thereby. According to some embodiments, each one of plurality of heaters 164 resides at least partially inside the well cavity 158 associated therewith.

As detailed above, aerosol generating device 100 is configured to provide controlled amount of cannabinoid concentrates upon effective vaporization. Aerosol generating device 100 disclosed herein is configured to provide cannabinoid aerosols from a variety of commercially available cannabinoid concentrates, as is not generally restricted to a specific cannabinoid concentrate. Specifically, aerosol generating device 100 is configured to hold and aerosolized viscous composition. As cannabinoid concentrates are inherently viscous and include concentrated amounts of cannabinoids, they fit to be aerosolized by aerosol generating device 100.

According to some embodiments, cannabinoid concentrate 160 comprises a cannabis plant extract. According to some embodiments, the cannabis extract is extracted using an organic solvent or supercritical carbon dioxide. According to some embodiments, the cannabis extract us extracted using an organic solvent. According to some embodiments, the cannabis extract us extracted using carbon dioxide. According to some embodiments, cannabinoid concentrate 160 is a full spectrum extract.

The term “full-spectrum extract”, often called whole plant extract, refers to a cannabis extract, which maintains the full profile of the cannabis plant. Full-spectrum extracts contain a variety of cannabinoids, including THC, tetrahydrocannabinolic acid (THCA), CBD, cannabidiolic acid (CBDA), cannabigerol (CBG), and cannabinol (CBN), as well as terpenes and other compounds such as flavonoids, proteins, phenols, sterols, and esters. Full-spectrum extracts are cannabinoid enriched, and are therefore, typically viscous.

The term “cannabinoid”, as used herein, includes all major and minor cannabinoids found in natural cannabis and hemp material that can be isolated from a natural source or reproduced by synthetic means. This includes delta-9-Tetrahydrocannabinol (THC), delta-9-tetrahydrocannabinolic acid (THCA), delta-8-Tetrahydrocannabinol, Cannabidiol (CBD), cannabidiolic acid (CBDA), cannabinol (CBN), cannabinolic acid (CBNA), tetrahydrocannabinovarin (THCV), cannabidivarin (CBDV), cannabigerol (CBG), cannabigerolic acid (CBGA) and cannabichromene (CBC).

According to some embodiments, each cannabinoid concentrate 160 within each one of well cavities 158 of plurality of wells 152 comprises a cannabinoid selected from tetrahydrocannabinol (THC), cannabidiol (CBD) or both.

According to some embodiments, each cannabinoid concentrate 160 within each one of well cavities 158 comprises at least 10% cannabinoids w/w. According to some embodiments, each cannabinoid concentrate 160 within each one of well cavities 158 comprises at least 15% cannabinoids w/w. According to some embodiments, each cannabinoid concentrate 160 within each one of well cavities 158 comprises at least 20% cannabinoids w/w. According to some embodiments, each cannabinoid concentrate 160 within each one of well cavities 158 comprises at least 25% cannabinoids w/w. According to some embodiments, each cannabinoid concentrate 160 within each one of well cavities 158 comprises at least 30% cannabinoids w/w. According to some embodiments, each cannabinoid concentrate 160 within each one of well cavities 158 comprises at least 40% cannabinoids w/w. According to some embodiments, each cannabinoid concentrate 160 within each one of well cavities 158 comprises at least 50% cannabinoids w/w. According to some embodiments, each cannabinoid concentrate 160 within each one of well cavities 158 comprises at least 60% cannabinoids w/w. According to some embodiments, each cannabinoid concentrate 160 within each one of well cavities 158 comprises at least 70% cannabinoids w/w.

According to some embodiments, each cannabinoid concentrate 160 within each one of well cavities 158 comprises at least 10% THC w/w. According to some embodiments, each cannabinoid concentrate 160 within each one of well cavities 158 comprises at least 15% THC w/w. According to some embodiments, each cannabinoid concentrate 160 within each one of well cavities 158 comprises at least 20% THC w/w. According to some embodiments, each cannabinoid concentrate 160 within each one of well cavities 158 comprises at least 25% THC w/w. According to some embodiments, each cannabinoid concentrate 160 within each one of well cavities 158 comprises at least 30% THC w/w. According to some embodiments, each cannabinoid concentrate 160 within each one of well cavities 158 comprises at least 40% THC w/w. According to some embodiments, each cannabinoid concentrate 160 within each one of well cavities 158 comprises at least 50% THC w/w. According to some embodiments, each cannabinoid concentrate 160 within each one of well cavities 158 comprises at least 60% THC w/w. According to some embodiments, each cannabinoid concentrate 160 within each one of well cavities 158 comprises at least 70% THC w/w.

According to some embodiments, each cannabinoid concentrate 160 within each one of well cavities 158 comprises at least 10% CBD w/w. According to some embodiments, each cannabinoid concentrate 160 within each one of well cavities 158 comprises at least 15% CBD w/w. According to some embodiments, each cannabinoid concentrate 160 within each one of well cavities 158 comprises at least 20% CBD w/w. According to some embodiments, each cannabinoid concentrate 160 within each one of well cavities 158 comprises at least 25% CBD w/w. According to some embodiments, each cannabinoid concentrate 160 within each one of well cavities 158 comprises at least 30% CBD w/w. According to some embodiments, each cannabinoid concentrate 160 within each one of well cavities 158 comprises at least 40% CBD w/w. According to some embodiments, each cannabinoid concentrate 160 within each one of well cavities 158 comprises at least 50% CBD w/w. According to some embodiments, each cannabinoid concentrate 160 within each one of well cavities 158 comprises at least 60% CBD w/w. According to some embodiments, each cannabinoid concentrate 160 within each one of well cavities 158 comprises at least 70% CBD w/w.

It is to be understood that the phrase—cannabinoid concentrate 160 within each one of well cavities 158 comprises at least X % of a specified material w/w—mean that value of the weight of the specified material divided by the weight of the cannabinoid concentrate 160 is at least X %.

According to some embodiments, the combined weight of cannabinoids within tray 151 is at least 10% w/w compared to the combined weight of cannabinoid concentrate 160 within all the well cavities 158. According to some embodiments, the combined weight of cannabinoids within tray 151 is at least 20% w/w compared to the combined weight of cannabinoid concentrate 160 within all the well cavities 158. According to some embodiments, the combined weight of cannabinoids within tray 151 is at least 30% w/w compared to the combined weight of cannabinoid concentrate 160 within all the well cavities 158. According to some embodiments, the combined weight of cannabinoids within tray 151 is at least 40% w/w compared to the combined weight of cannabinoid concentrate 160 within all the well cavities 158. According to some embodiments, the combined weight of cannabinoids within tray 151 is at least 50% w/w compared to the combined weight of cannabinoid concentrate 160 within all the well cavities 158. According to some embodiments, the combined weight of cannabinoids within tray 151 is at least 60% w/w compared to the combined weight of cannabinoid concentrate 160 within all the well cavities 158. According to some embodiments, the combined weight of cannabinoids within tray 151 is at least 70% w/w compared to the combined weight of cannabinoid concentrate 160 within all the well cavities 158.

According to some embodiments, the combined weight of THC within tray 151 is at least 10% w/w compared to the combined weight of cannabinoid concentrate 160 within all the well cavities 158. According to some embodiments, the combined weight of THC within tray 151 is at least 20% w/w compared to the combined weight of cannabinoid concentrate 160 within all the well cavities 158. According to some embodiments, the combined weight of THC within tray 151 is at least 30% w/w compared to the combined weight of cannabinoid concentrate 160 within all the well cavities 158. According to some embodiments, the combined weight of THC within tray 151 is at least 40% w/w compared to the combined weight of cannabinoid concentrate 160 within all the well cavities 158. According to some embodiments, the combined weight of THC within tray 151 is at least 50% w/w compared to the combined weight of cannabinoid concentrate 160 within all the well cavities 158. According to some embodiments, the combined weight of THC within tray 151 is at least 60% w/w compared to the combined weight of cannabinoid concentrate 160 within all the well cavities 158. According to some embodiments, the combined weight of THC within tray 151 is at least 70% w/w compared to the combined weight of cannabinoid concentrate 160 within all the well cavities 158.

According to some embodiments, the combined weight of CBD within tray 151 is at least 10% w/w compared to the combined weight of cannabinoid concentrate 160 within all the well cavities 158. According to some embodiments, the combined weight of CBD within tray 151 is at least 20% w/w compared to the combined weight of cannabinoid concentrate 160 within all the well cavities 158. According to some embodiments, the combined weight of CBD within tray 151 is at least 30% w/w compared to the combined weight of cannabinoid concentrate 160 within all the well cavities 158. According to some embodiments, the combined weight of CBD within tray 151 is at least 40% w/w compared to the combined weight of cannabinoid concentrate 160 within all the well cavities 158. According to some embodiments, the combined weight of CBD within tray 151 is at least 50% w/w compared to the combined weight of cannabinoid concentrate 160 within all the well cavities 158. According to some embodiments, the combined weight of CBD within tray 151 is at least 60% w/w compared to the combined weight of cannabinoid concentrate 160 within all the well cavities 158. According to some embodiments, the combined weight of CBD within tray 151 is at least 70% w/w compared to the combined weight of cannabinoid concentrate 160 within all the well cavities 158.

According to some embodiments, each cannabinoid concentrate contained within one of the cavities comprises 0.25 to 5 milligrams of cannabinoids. According to some embodiments, each cannabinoid concentrate contained within one of the cavities comprises 0.25 to 0.5 milligrams of cannabinoids. According to some embodiments, each cannabinoid concentrate contained within one of the cavities comprises 0.5 to 1 milligrams of cannabinoids. According to some embodiments, each cannabinoid concentrate contained within one of the cavities comprises 1 to 2 milligrams of cannabinoids. According to some embodiments, each cannabinoid concentrate contained within one of the cavities comprises 0.5 to 1 milligrams of cannabinoids. According to some embodiments, each cannabinoid concentrate contained within one of the cavities comprises 1 to 2 milligrams of cannabinoids. According to some embodiments, each cannabinoid concentrate contained within one of the cavities comprises 2 to 3 milligrams of cannabinoids. According to some embodiments, each cannabinoid concentrate contained within one of the cavities comprises 3 to 5 milligrams of cannabinoids.

According to some embodiments, each cannabinoid concentrate contained within one of the cavities comprises at least 0.25 milligrams of cannabinoids. According to some embodiments, each cannabinoid concentrate contained within one of the cavities comprises at least 0.5 milligrams of cannabinoids. According to some embodiments, each cannabinoid concentrate contained within one of the cavities comprises at least 1 milligram of cannabinoids. According to some embodiments, each cannabinoid concentrate contained within one of the cavities comprises at least 2 milligrams of cannabinoids. According to some embodiments, each cannabinoid concentrate contained within one of the cavities comprises at least 3 milligrams of cannabinoids.

According to some embodiments, each cannabinoid concentrate contained within one of the cavities comprises no more than 5 milligrams of cannabinoids. According to some embodiments, each cannabinoid concentrate contained within one of the cavities comprises no more than 4 milligrams of cannabinoids. According to some embodiments, each cannabinoid concentrate contained within one of the cavities comprises no more than 3 milligrams of cannabinoids. According to some embodiments, each cannabinoid concentrate contained within one of the cavities comprises no more than 2.5 milligrams of cannabinoids. According to some embodiments, each cannabinoid concentrate contained within one of the cavities comprises no more than 2 milligrams of cannabinoids.

According to some embodiments, each cannabinoid concentrate contained within one of the cavities comprises at least 0.25 milligrams of THC. According to some embodiments, each cannabinoid concentrate contained within one of the cavities comprises at least 0.5 milligrams of THC. According to some embodiments, each cannabinoid concentrate contained within one of the cavities comprises at least 1 milligram of THC. According to some embodiments, each cannabinoid concentrate contained within one of the cavities comprises at least 2 milligrams of THC. According to some embodiments, each cannabinoid concentrate contained within one of the cavities comprises at least 3 milligrams of THC.

According to some embodiments, each cannabinoid concentrate contained within one of the cavities comprises no more than 5 milligrams of THC. According to some embodiments, each cannabinoid concentrate contained within one of the cavities comprises no more than 4 milligrams of THC. According to some embodiments, each cannabinoid concentrate contained within one of the cavities comprises no more than 3 milligrams of THC. According to some embodiments, each cannabinoid concentrate contained within one of the cavities comprises no more than 2.5 milligrams of THC. According to some embodiments, each cannabinoid concentrate contained within one of the cavities comprises no more than 2 milligrams of THC.

According to some embodiments, each cannabinoid concentrate contained within one of the cavities comprises at least 0.25 milligrams of CBD. According to some embodiments, each cannabinoid concentrate contained within one of the cavities comprises at least 0.5 milligrams of CBD. According to some embodiments, each cannabinoid concentrate contained within one of the cavities comprises at least 1 milligram of CBD. According to some embodiments, each cannabinoid concentrate contained within one of the cavities comprises at least 2 milligrams of CBD. According to some embodiments, each cannabinoid concentrate contained within one of the cavities comprises at least 3 milligrams of CBD.

According to some embodiments, each cannabinoid concentrate contained within one of the cavities comprises no more than 5 milligrams of CBD. According to some embodiments, each cannabinoid concentrate contained within one of the cavities comprises no more than 4 milligrams of CBD. According to some embodiments, each cannabinoid concentrate contained within one of the cavities comprises no more than 3 milligrams of CBD. According to some embodiments, each cannabinoid concentrate contained within one of the cavities comprises no more than 2.5 milligrams of CBD. According to some embodiments, each cannabinoid concentrate contained within one of the cavities comprises no more than 2 milligrams of CBD.

According to some embodiments, each cannabinoid concentrate contained within one of the cavities comprises 0.25 to 5 milligrams of a cannabinoid selected from tetrahydrocannabinol (THC), cannabidiol (CBD) or both.

According to some embodiments, the total mass of the cannabinoid concentrate within tray 151 is in the range of 40 to 500 milligrams. According to some embodiments, the total mass of the cannabinoid concentrate within tray 151 is in the range of 40 to 60 milligrams. According to some embodiments, the total mass of the cannabinoid concentrate within tray 151 is in the range of 60 to 100 milligrams. According to some embodiments, the total mass of the cannabinoid concentrate within tray 151 is in the range of 100 to 150 milligrams. According to some embodiments, the total mass of the cannabinoid concentrate within tray 151 is in the range of 100 to 200 milligrams. According to some embodiments, the total mass of the cannabinoid concentrate within tray 151 is in the range of 200 to 500 milligrams. According to some embodiments, the total mass of the cannabinoid concentrate within tray 151 is in the range of 250 to 500 milligrams.

According to some embodiments, each cannabinoid concentrate 160 contained within one of well cavities 158 of plurality of wells 152 has mass in the range of 0.5 to 5 milligrams. According to some embodiments, each cannabinoid concentrate 160 contained within one of well cavities 158 of plurality of wells 152 has mass in the range of 0.5 to 1.5 milligrams. According to some embodiments, each cannabinoid concentrate 160 contained within one of well cavities 158 of plurality of wells 152 has mass in the range of 1.5 to 3.5 milligrams. According to some embodiments, each cannabinoid concentrate 160 contained within one of well cavities 158 of plurality of wells 152 has mass in the range of 2 to 5 milligrams.

According to some embodiments, each cannabinoid concentrate 160 contained within one of well cavities 158 of plurality of wells 152 has mass of at least 0.5 milligrams. According to some embodiments, each cannabinoid concentrate 160 contained within one of well cavities 158 of plurality of wells 152 has mass of at least 1 milligram. According to some embodiments, each cannabinoid concentrate 160 contained within one of well cavities 158 of plurality of wells 152 has mass of at least 2 milligrams. According to some embodiments, each cannabinoid concentrate 160 contained within one of well cavities 158 of plurality of wells 152 has mass of at least 2.5 milligrams.

According to some embodiments, each cannabinoid concentrate 160 contained within one of well cavities 158 of plurality of wells 152 has mass of no more than 5 milligrams. According to some embodiments, each cannabinoid concentrate 160 contained within one of well cavities 158 of plurality of wells 152 has mass of no more than 4.5 milligrams. According to some embodiments, each cannabinoid concentrate 160 contained within one of well cavities 158 of plurality of wells 152 has mass of no more than 4 milligrams. According to some embodiments, each cannabinoid concentrate 160 contained within one of well cavities 158 of plurality of wells 152 has mass of no more than 3.5 milligrams. According to some embodiments, each cannabinoid concentrate 160 contained within one of well cavities 158 of plurality of wells 152 has mass of no more than 3 milligrams.

According to some embodiments, each of plurality of wells 152 comprises cannabinoid concentrate 160 within its cavity 158. According to some embodiments, each one of plurality of wells 152 comprises the same amount of cannabinoid concentrate 160 cannabinoid concentrate within it cavity 158 as the other wells 152.

According to some embodiments, each one of cavities 158 has a volume in the range of 0.5-10 microliters. According to some embodiments, each one of cavities 158 has a volume in the range of 1-5 microliters.

According to some embodiments, each one of cavities 158 has a volume of at least 0.5 microliters. According to some embodiments, each one of cavities 158 has a volume of at least 1 microliter. According to some embodiments, each one of cavities 158 has a volume of at least 2 microliters. According to some embodiments, each one of cavities 158 has a volume of at least 3 microliters. According to some embodiments, each one of cavities 158 has a volume of at least 4 microliters. According to some embodiments, each one of cavities 158 has a volume of at least 5 microliters.

According to some embodiments, each one of cavities 158 has a volume of no more than 10 microliters. According to some embodiments, each one of cavities 158 has a volume of no more than 9 microliters. According to some embodiments, each one of cavities 158 has a volume of no more than 8 microliters. According to some embodiments, each one of cavities 158 has a volume of no more than 7 microliters. According to some embodiments, each one of cavities 158 has a volume of no more than 6 microliters. According to some embodiments, each one of cavities 158 has a volume of no more than 5 microliters.

According to some embodiments, each of the plurality of well has the same volume as the other wells.

As detailed herein aerosol generating device 100 is especially designed for aerosolization of cannabinoid concentrates. In particular, cannabinoid concentrates 160 are contained within partially open wells 152 (each one of plurality of wells 152 has an open side 154). As cannabinoid concentrate 160 are viscous, this does not pose a risk of spillage of the cannabinoid concentrate 160 from the well cavities 158 associated therewith. Thus, aerosol generating device 100 may be a hand held device, according to some embodiments. Such hand-held devices are required to sustain occasional impact caused by shaking an moving the device. As concentrates are viscous they were found to remain within their cavities without spilling out.

According to some embodiments, the cannabinoid concentrate has a viscosity of at least 1000 mPa·s. According to some embodiments, the cannabinoid concentrate has a viscosity of at least 1500 mPa·s. According to some embodiments, the cannabinoid concentrate has a viscosity of at least 2000 mPa·s. According to some embodiments, the cannabinoid concentrate has a viscosity of at least 2500 mPa·s. According to some embodiments, the cannabinoid concentrate has a viscosity of at least 3000 mPa·s. According to some embodiments, the cannabinoid concentrate has a viscosity of at least 4000 mPa·s. According to some embodiments, the cannabinoid concentrate has a viscosity of at least 5000 mPa·s.

The unit mPa·s, millipascal-second, is conventional unit for measuring viscosity. It is equal to 1/100 Poise, or centipoise, poise being the standard centimeter-gram-second system viscosity unit. Exemplary values are: water −1 mPa·s, mercury 1.5 mPa·s, whole milk −2.1 mPa·s. viscous materials, such as honey and peanut butter have viscosities of 1000 mPa·s or more.

Another physical property characteristic to cannabinoids is their tendency to evaporate upon heating and to form aerosol upon the vapor cooling.

Specifically, according to some embodiments, each one of plurality of heaters 164 is configured to elevate the temperature a respective well 152 of plurality of wells 152, thereby to produce cannabinoid vapor from the respective cannabinoid concentrate 160.

It is to be understood that the phrase “respective” has the same meaning as “associated with” as presented above. Specifically, when a specific heater 164 is positioned and configured to elevated the temperature of this specific well 152, its specific well cavity 158 and the specific cannabinoid concentrate 160 within the specific well cavity 158—all these elements are respective one to the other.

Cannabinoid concentrates 160 are contained within well cavities 158, wherein upon heating a cannabinoid concentrate 160 within a well cavity 158, it is vaporized, according to some embodiments. As each one of plurality of wells 152 has a closed face 156 and an open side 154, the flow of cannabinoid vapor formed upon said vaporization, is directed out of open side 154. As open side 154 faces outlet 102, the vaporized cannabinoids are flowing in the outlet 102 direction. However, during the flow from well cavity 158 through open side 154 in the outlet 102 direction, the cannabinoid vapor is cooled and condenses into droplets containing the cannabinoids. The liquid-gas dispersion of cannabinoids and air is in the form of an aerosol, due to the small droplets of cannabinoids within the air matrix, according to some embodiments. Thus, the vaporized cannabinoids exit aerosol generating device 100 through outlet 102 as an aerosol, according to some embodiments. It is to be understood that the cannabinoid aerosol may include different natural compounds in either the gas, liquid or solid states, as sometimes not all the material timely condenses.

As detailed herein aerosol generating device 100 is configured to provide an effective high dose of aerosolized cannabinoid concentrate 160 to the lungs of a user. Without wishing to be bound by any theory or mechanism of action, high dosage of cannabinoids reaches the lungs by inhaling aerosolized cannabinoid concentrate 160 using aerosol generating device 100 an electronic cigarette has small aerosol droplets, having MMAD within the range of 0.1 to 1 microns. It is noted that such small droplets are maintained even at aerosol produced with high cannabinoid concentrations. Thus, high cannabinoid concentrations can be inhaled and reach the lungs using aerosol generating device 100.

According to some embodiments, the aerosol composition comprises droplets having a mass median aerodynamic diameter (MMAD) of at most 5 microns. According to some embodiments, the aerosol composition comprises droplets having a mass median aerodynamic diameter (MMAD) of at most 4 microns. According to some embodiments, the aerosol composition comprises droplets having a mass median aerodynamic diameter (MMAD) of at most 3 microns. According to some embodiments, the aerosol composition comprises droplets having a mass median aerodynamic diameter (MMAD) of at most 2 microns. According to some embodiments, the aerosol composition comprises droplets having a mass median aerodynamic diameter (MMAD) of at most 1 microns. According to some embodiments, the aerosol composition comprises droplets having a mass median aerodynamic diameter (MMAD) of at most 0.9 microns. According to some embodiments, the aerosol composition comprises droplets having a mass median aerodynamic diameter (MMAD) of at most 0.8 microns. According to some embodiments, the aerosol composition comprises droplets having a mass median aerodynamic diameter (MMAD) of at most 0.7 microns. According to some embodiments, the aerosol composition comprises droplets having a mass median aerodynamic diameter (MMAD) of at most 0.6 microns. According to some embodiments, the aerosol composition comprises droplets having a mass median aerodynamic diameter (MMAD) of at most 0.5 microns. According to some embodiments, the aerosol composition comprises droplets having a mass median aerodynamic diameter (MMAD) in the range of 0.1 to 1 microns.

It was surprisingly found that aerosolization of cannabinoid concentrate 160 using aerosol generating device 100 as disclosed herein, results in droplets having a mass median aerodynamic diameter (MMAD) sufficiently small so as to reach the lungs, rather than precipitate on their way thereto. The small droplets reaching the lungs enable efficient respiratory delivery of the cannabinoid(s). This is an overall advantage as maximizing the delivery of cannabinoid(s) to the lungs, while minimizing its deposition in the mouth and throat are considered highly beneficial.

The terms ‘droplet size’ and ‘mass median aerodynamic diameter’, also known as MMAD, as used herein are interchangeable. MMAD is commonly considered as the median particle diameter by mass. MMAD may be evaluated by plotting droplet size vs. the cumulative mass fraction (%) in the aerosol. MMAD may then be determined according to the interpolated droplet size corresponding to the point, where the cumulative mass fraction is 50%. This points represent the estimated values of particle sizes, above which the droplets are responsible to half to masses and below which the droplets are responsible to the other halves, in each solution.

Another feature of aerosol generating device 100, which impacts the formation of aerosol is its shape, or in particular, the shape of a mouthpiece 106 thereof.

According to some embodiments, aerosol generating device 100 comprises a mouthpiece 106 as shown in FIGS. 1 and 2. According to some embodiments, the mouthpiece 106 extends between outlet 102 and a proximal mouthpiece side 107. According to some embodiments, proximal mouthpiece side 107 faces the open side 154 of each one of plurality of heaters 164. According to some embodiments, mouthpiece 106 is tapering towards outlet 102. According to some embodiments, mouthpiece 106 is tapering from proximal mouthpiece side 107 towards outlet 102. According to some embodiments, upon production of the cannabinoid vapor, the vapor flows from open side 154 through the proximal face and out the outlet 102. According to some embodiments, during said flowing the vapor at least partially condenses to produce a cannabinoid aerosol.

It is understood that the tapering structure of the mouthpiece 106 is effective the cannabinoid vapor flow and velocity, and thus the formation of aerosol therefrom. It was found that a tapering structure results in the formation of aerosol.

One of the advantages of the present aerosol generating device 100 is that it enables dosing and better controlling the amount of consumed aerosol in a period of time (e.g. per use, per day, per week etc.). as detailed herein, this is enabled by the separate control of processing unit 204 over each one of a plurality of known discrete portions of cannabinoid concentrate 160.

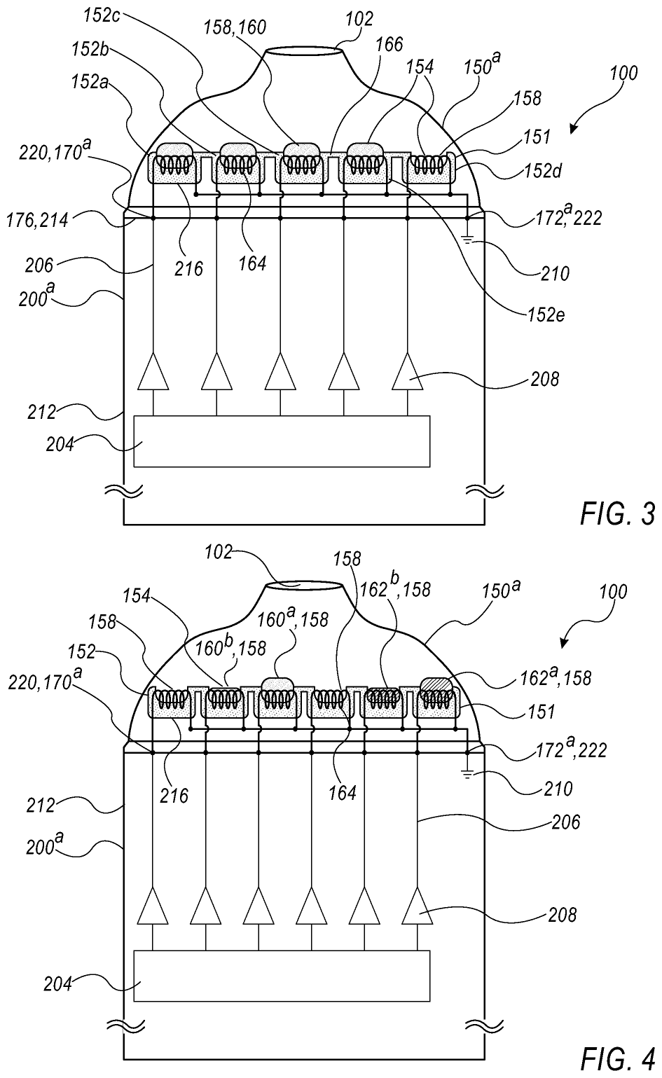

Thus, it may be preferable, according to some embodiments, that the controlled elevation of temperature within each well 152 is substantially confined to the specific well 152. Otherwise there may be a risk of affecting adjacent wells 152 (e.g. well 152a and well 152b shown in FIG. 3). Such confinement may be achieved, according to some embodiments, through constructing tray 151 with thermally isolating material.

According to some embodiments, the tray 151 comprises of a thermally insulating material. According to some embodiments, the tray 151 is made of a thermally insulating material.

According to some embodiments, each one of plurality of wells 152 is thermally isolated from the other wells 152.

According to some embodiments, tray 151 comprises plurality of wells 152 and a plurality of inter-well joints 166. According to some embodiments, each one of inter-well joints 166 is inter-connecting between two adjacent well 152. According to some embodiments, each one of inter-well joints 166 is made of a thermally insulating material.

As detailed above, aerosol generating device 100 presented in FIGS. 1 and 2 (as well as aerosol generating device 100 presented in FIGS. 3 and 4) comprises two connectable parts—cartridge 150^{a} and controlling member 200^{a}. According to some embodiments, controlling member 200^{a} is connectable to cartridge 150^{a}. According to some embodiments, upon connection aerosol generating device 100 is assembled.

According to some embodiments, upon assembly of aerosol generating device 100, processing unit 204 forms an electric contact with each one of plurality of heaters 164 separately.

As further detailed above, controlling member 200^{a} comprises controlling member housing 212, which houses processing unit 204, according to some embodiments. According to some embodiments, controlling member housing 212 comprises a controlling member housing proximal face 214 and a controlling member housing distal face 216. According to some embodiments, controlling member housing proximal face 214 is facing cartridge 150^{a} when aerosol generating device 100 is assembled. According to some embodiments, controlling member housing distal face 216 is located distally from facing cartridge 150^{a} when aerosol generating device 100 is assembled. According to some embodiments, controlling member housing proximal face 214 and controlling member housing distal face 216 are at opposite sides of controlling member 200^{a}.

According to some embodiments, cartridge 150^{a} comprises a cartridge distal face 176 and a cartridge proximal side 174. According to some embodiments, cartridge distal face 176 is facing controlling member 200^{a} when aerosol generating device 100 is assembled. According to some embodiments, cartridge proximal side 174 is located distally from controlling member 200^{a} when aerosol generating device 100 is assembled. According to some embodiments, cartridge proximal side 174 is located adjacent to outlet 102. According to some embodiments, cartridge distal face 176 and cartridge proximal side 174 are at opposite sides of cartridge 150^{a}.

According to some embodiments, upon assembly of aerosol generating device 100 controlling member housing proximal face 214 is facing cartridge distal face 176. According to some embodiments, upon assembly of aerosol generating device 100 controlling member housing proximal face 214 is contacting cartridge distal face 176.

According to some embodiments, controlling member 200^{a} comprises a reader 218. According to some embodiments, controlling member 200^{a} comprises a reader 218 adjacent to controlling member housing proximal face 214. According to some embodiments, controlling member 200^{a} comprises a reader 218 at controlling member housing proximal face 214.

According to some embodiments, cartridge 150^{a} comprises an identifier 168. According to some embodiments, cartridge 150^{a} comprises an identifier 168 adjacent to cartridge distal face 176. According to some embodiments, cartridge 150^{a} comprises an identifier 168 at cartridge distal face 176.

According to some embodiments, identifier 168 is positioned to face reader 218 upon assembly of aerosol generating device 100. According to some embodiments, reader 218 is configured to identify identifier 168. According to some embodiments, reader 218 is further configured to send identification signals indicative of the identification to processing unit 204.

Specifically, different cartridges 150^{a} may be provided with different dosages and compositions within the well cavities 158 of their trays 151. It is an important feature of the presently provided aerosol generating device 100 to enable controlled dosing of the consumed cannabinoid compositions. Therefore, it may be important, in cases that different cartridges 150^{a} are manufactured, that processing unit 204 recognizes the specification of the compositions contained within each cartridge 150^{a}. In example, processing unit 204 may operate in a mode, which consumes well 152 after well 152, as further elaborated below. In such case, processing unit 204 should be able to recognize when cannabinoid concentrate 160 in a specific well is consumed. However, different cartridge 150^{a} may have different types or amounts of cannabinoid concentrates 160 or different volumes of well cavities 158. Since this affects the consumed amount, it would affect the controlling operation of processing unit 204 over plurality of heaters 164. Therefore, when different cartridges 150^{a} are provided, a distinctive identification may be required, according to some embodiments. Since cartridge 150^{a} is intended to be disposable, it may include an identifier 168, such as a barcode, and the durable controlling member 200^{a} may include a reader 218, such as a barcode reader.

According to some embodiments, the identification comprises information about contents within each well cavity 158 of plurality of wells 152. According to some embodiments, the identification comprises information about the volume of each well cavity 158 of plurality of wells 152. According to some embodiments, the identification comprises information about each one of plurality of wells 152, the information comprises the temperature and duration required for full vaporization of cannabinoid concentrate 160 contained within each one of plurality of wells 152.

According to some embodiments, identifier 168 is a barcode and the reader is a barcode reader. According to some embodiments, identifier 168 is a QR code and the reader is a QR code.

It is to be understood that although identifier 168 and reader 218 are drawn only in some of the figures, these elements may be present in any of aerosol generating devices 100 detailed herein.

The heaters 164 of the present aerosol generating device 100 should have physical and electric properties to be able to vaporize cannabinoid concentrates 160 within well cavities 158 effectively. According to some embodiments, each one of plurality of heaters 164 is configured to effectively evaporate each corresponding cannabinoid concentrate 160 within the corresponding well cavity 158. According to some embodiments, each one of plurality of heaters 164 is configured to effectively evaporate each corresponding cannabinoid concentrate 160 within the corresponding well cavity 158 within 10 seconds. According to some embodiments, each one of plurality of heaters 164 is configured to effectively evaporate each corresponding cannabinoid concentrate 160 within the corresponding well cavity 158 within 8 seconds. According to some embodiments, each one of plurality of heaters 164 is configured to effectively evaporate each corresponding cannabinoid concentrate 160 within the corresponding well cavity 158 within 6 seconds. According to some embodiments, each one of plurality of heaters 164 is configured to effectively evaporate each corresponding cannabinoid concentrate 160 within the corresponding well cavity 158 within 5 seconds. According to some embodiments, each one of plurality of heaters 164 is configured to effectively evaporate each corresponding cannabinoid concentrate 160 within the corresponding well cavity 158 within 4 seconds. According to some embodiments, each one of plurality of heaters 164 is configured to effectively evaporate each corresponding cannabinoid concentrate 160 within the corresponding well cavity 158 within 3 seconds. According to some embodiments, each one of plurality of heaters 164 is configured to effectively evaporate each corresponding cannabinoid concentrate 160 within the corresponding well cavity 158 within 2 seconds. According to some embodiments, each one of plurality of heaters 164 is configured to effectively evaporate each corresponding cannabinoid concentrate 160 within the corresponding well cavity 158 within 1 second.

The terms “effective evaporation” and “substantial evaporation” are interchangeable and are intended to mean that at least 50%, at least 60%, at least 70%, at least 75%, at least 80%, at least 85%, at least 90%, at least 95%, least 97%, least 98%, least 99%, least 99.5% or least 99.9% of a composition is transformed from liquid or solid to gaseous state.

According to some embodiments, each one of plurality of heaters 164 has a total resistance in the range of 0.2 to 2 Ohms. According to some embodiments, each one of plurality of heaters 164 has a total resistance in the range of 0.2 to 4 Ohms. According to some embodiments, each one of plurality of heaters 164 has a total resistance in the range of 0.4 to 0.8 Ohms. According to some embodiments, each one of plurality of heaters 164 has a total resistance in the range of 0.8 to 1.4 Ohms. According to some embodiments, each one of plurality of heaters 164 has a total resistance in the range of 1.4 to 2 Ohms.

According to some embodiments, each one of plurality of heaters 164 has a total resistance of at least 0.2 Ohms. According to some embodiments, each one of plurality of heaters 164 has a total resistance of at least 0.3 Ohms. According to some embodiments, each one of plurality of heaters 164 has a total resistance of at least 0.4 Ohms. According to some embodiments, each one of plurality of heaters 164 has a total resistance of at least 0.5 Ohms. According to some embodiments, each one of plurality of heaters 164 has a total resistance of at least 0.7 Ohms. According to some embodiments, each one of plurality of heaters 164 has a total resistance of at least 0.8 Ohms. According to some embodiments, each one of plurality of heaters 164 has a total resistance of at least 0.9 Ohms. According to some embodiments, each one of plurality of heaters 164 has a total resistance of at least 1 Ohm.

According to some embodiments, each one of plurality of heaters 164 has a total resistance of no more than 2 Ohms. According to some embodiments, each one of plurality of heaters 164 has a total resistance of no more than 1.8 Ohms. According to some embodiments, each one of plurality of heaters 164 has a total resistance of no more than 1.6 Ohms. According to some embodiments, each one of plurality of heaters 164 has a total resistance of no more than 1.4 Ohms. According to some embodiments, each one of plurality of heaters 164 has a total resistance of no more than 1.2 Ohms. According to some embodiments, each one of plurality of heaters 164 has a total resistance of no more than 1 Ohm.

According to some embodiments, one or more of plurality of heaters 164 are operated using an induction coil (not shown). Particularly, in such embodiments, an electric current is provided to the induction coil and the magnetic field generated by the induction coil produces a current within the respective heater 164, thereby generating heat.

According to some embodiments, one or more of plurality of heaters 164 are operated using a laser (not shown). Particularly, in such embodiments, a laser beam is directed at either an element of the respective heater 164, which heats up responsive to the laser beam, or at a respective well 152, which heats up responsive to the laser beam.

According to some embodiments, each one of plurality of heaters 164 is configured to provide an energy output in the range of 1 to 50 Watts. cording to some embodiments, each one of plurality of heaters 164 is configured to provide an energy output in the range of 1 to 50 Watts.

According to some embodiments, each one of plurality of heaters 164 is configured to provide an energy output in the range of 1 to 5 Watts. According to some embodiments, each one of plurality of heaters 164 is configured to provide an energy output in the range of 1 to 50 Watts. According to some embodiments, each one of plurality of heaters 164 is configured to provide an energy output in the range of 5 to 20 Watts. According to some embodiments, each one of plurality of heaters 164 is configured to provide an energy output in the range of 10 to 30 Watts. According to some embodiments, each one of plurality of heaters 164 is configured to provide an energy output in the range of 25 to 50 Watts.

According to some embodiments, each one of plurality of heaters 164 is configured to provide an energy of at least 1 Watt. According to some embodiments, each one of plurality of heaters 164 is configured to provide an energy of at least 2 Watts. According to some embodiments, each one of plurality of heaters 164 is configured to provide an energy of at least 4 Watts. According to some embodiments, each one of plurality of heaters 164 is configured to provide an energy of at least 6 Watts. According to some embodiments, each one of plurality of heaters 164 is configured to provide an energy of at least 8 Watts. According to some embodiments, each one of plurality of heaters 164 is configured to provide an energy of at least 10 Watts. According to some embodiments, each one of plurality of heaters 164 is configured to provide an energy of at least 15 Watts. According to some embodiments, each one of plurality of heaters 164 is configured to provide an energy of at least 20 Watts. According to some embodiments, each one of plurality of heaters 164 is configured to provide an energy of at least 25 Watts.

According to some embodiments, each one of plurality of heaters 164 is configured to provide an energy of no more than 50 Watts. According to some embodiments, each one of plurality of heaters 164 is configured to provide an energy of at on more than 45 Watts. According to some embodiments, each one of plurality of heaters 164 is configured to provide an energy of at on more than 40 Watts. According to some embodiments, each one of plurality of heaters 164 is configured to provide an energy of at on more than 35 Watts. According to some embodiments, each one of plurality of heaters 164 is configured to provide an energy of at on more than 30 Watts. According to some embodiments, each one of plurality of heaters 164 is configured to provide an energy of at on more than 25 Watts.

According to some embodiments, processing unit 204 is electrically connected to each one of plurality of heaters 164 through an electric driver 208.

According to some embodiments, electric driver 208 is selected from the group consisting of an electric switch and a transistor. According to some embodiments, electric driver 208 is configured to control the wattage and/or current provided to each one of plurality of heaters 164.

FIGS. 1-2 also show a plurality of electric wires 206 connecting between processing unit 204 and each one of plurality of heaters 164 (through electric driver 208). Specifically, according to some embodiments, cartridge 150^{a} has a plurality of cartridge electric contacts 170^{a}, each connected electrically to one of plurality of heaters 164. According to some embodiments, plurality of cartridge electric contacts 170^{a} are located at cartridge distal face 176. Also, according to some embodiments, controlling member 200^{a} has a plurality of controlling member contacts 220. According to some embodiments, plurality of controlling member contacts 220 are located at controlling member housing proximal face 214. According to some embodiments, upon assembly plurality of controlling member contacts 220 are contacting plurality of cartridge electric contacts 170^{a}. According to some embodiments, upon assembly of aerosol generating device 100, each one of plurality of controlling member contacts 220 is contacting one of plurality of cartridge electric contacts 170^{a}. According to some embodiments, upon assembly of aerosol generating device 100, each one of plurality of controlling member contacts 220 is electrically connected to one of plurality of cartridge electric contacts 170^{a}.

Another element usually required for electronic application, and shown in the figures is ground 210. Ground are occasionally used for safety reasons, as can be appreciated by the skilled in the art. According to some embodiments, controlling member 200^{a} comprises ground 210, electrically connected to cartridge ground contact 172^{a}. According to some embodiments, cartridge ground contact 172^{a} is located at controlling member housing proximal face 214. According to some embodiments, cartridge 150^{a} comprises controlling member ground contact 222. According to some embodiments, controlling member ground contact 222 is electrically connected to each one of plurality of heaters 164. According to some embodiments, controlling member ground contact 222 is located at cartridge distal face 176. According to some embodiments, upon assembly of aerosol generating device 100, controlling member ground contact 222 is electrically connected to cartridge ground contact 172^{a}, thereby electrically associating between ground 210 and each one of plurality of heaters 164.

According to some embodiments, each one of plurality of heaters 164 is configured to elevate the temperature of the well 152 heated thereby to a predetermined temperature. According to some embodiments, each one of plurality of heaters 164 is configured to elevate the temperature of the well cavity 158 heated thereby to a predetermined temperature. According to some embodiments, each one of plurality of heaters 164 is configured to elevate the temperature of the cannabinoid concentrate 160 heated thereby to a predetermined temperature.

According to some embodiments, processing unit 204 is configured to separately operate each one of plurality of heaters 164, thereby to elevate the temperature within each corresponding well 152 individually to the predetermined temperature. According to some embodiments, processing unit 204 is configured to separately operate each one of plurality of heaters 164, thereby to elevate the temperature within each corresponding well cavity 158 individually to the predetermined temperature. According to some embodiments, processing unit 204 is configured to separately operate each one of plurality of heaters 164, thereby to elevate the temperature within each corresponding cannabinoid concentrate 160 individually to the predetermined temperature.

Specifically, it is to be understood that the control over the temperature may be effecting the rate of cannabinoid concentrate vaporization, thereby controlling the dosing, according to some embodiments.

According to some embodiments, upon being heated to the predetermined temperature by the respective heater 164, cannabinoid concentrate 160 is being evaporated to form cannabinoid vapor at a predetermined rate. Specifically, it is to be understood that the predetermined vaporization rate is the mass of cannabinoid being evaporated per time unit. This may be measured e.g. by the weight loss of cannabinoid concentrate 160 as a function of time. For example, if a well cavity 158 contains 100 milligrams of cannabinoid concentrate 160, and processing unit 204 is operating the corresponding heater 164 for 1 second to evaporate part of cannabinoid concentrate 160, wherein upon the evaporation 50 milligrams of cannabinoid concentrate 160 remains inside well cavity 158, the evaporation rate is 50 milligrams per second. It is further to be understood that the predetermined vaporization rate is dictated mainly by the amount of cannabinoid concentrate 160 within each well 152, the temperature at which cannabinoid concentrate 160 is heated, and the cannabinoid concentrate 160 chemical composition (i.e. the chemical components composition and ratio, and their inherent tendency to vaporize), according to some embodiments. The amount of cannabinoid concentrate 160 in each well 152, in its turn, may be determined by the well cavity 158 volume and the well filling proportion.

The present aerosol generating device 100 may enable both control over the vaporization rate and monitoring the total amount of vaporized cannabinoids, according to some embodiments. According to some embodiments, processing unit 204 is configured to control the predetermined vaporization rate. According to some embodiments, processing unit 204 is configured to control the predetermined vaporization rate upon instructions from a user of aerosol generating device 100. This is further elaborated below, when user interfaces are discussed herein.

It is further to be understood that monitoring the total amount of vaporized cannabinoids by processing unit 204 may be enabled by a combination of parameters, according to some embodiments. Such parameters may include the vaporization rate and the vaporization time period, according to some embodiments. For example, vaporization of cannabinoid concentrate 160 for 5 second at a rate of 0.3 milligrams per second will result in a total vaporization of 1.5 milligrams cannabinoids. Processing unit 204 may include instructions to cease heating wells(s) 152 upon processing unit 204 calculating a consumption of a specific amount of cannabinoids, according to some embodiments. For example, processing unit 204 may receive instructions from a physician (via a user interface as detailed below) to cease operation of plurality of heaters 164 upon consumption of 3 milligrams cannabinoid. In this example, the operation of heaters 164 is postponed for a specified time (e.g. for a daily dose of 3 milligrams/day, it will postpone for 24 hours), and thereafter enable 3 more milligrams to be consumed (e.g. two 5-second puffs, as described above).

According to some embodiments, processing unit 204 is configured to receive instructions to cease operation of plurality of heaters 164. According to some embodiments, processing unit 204 is configured to receive instructions to postpone operation of plurality of heaters 164 for a predetermined postponement period upon the total amount of vaporized cannabinoids being equal to a predetermined threshold. According to some embodiments, processing unit 204 comprises instructions to postpone operation of plurality of heaters 164 for a predetermined postponement period upon the total amount of vaporized cannabinoids being equal to a predetermined threshold. According to some embodiments, the predetermined postponement period is selected from the group consisting of 2-4 hours, 4-8 hours, 8-24 hours, 1-2 days and 1-7 days. Each possibility represents a separate embodiment. According to some embodiments, the predetermined postponement period is in the range of 8 hours to 2 days. According to some embodiments, the total amount of vaporized cannabinoids is calculated by processing unit 204. According to some embodiments, the calculation is a function of the evaporation time and evaporation rate of each one of plurality of heaters 164 in the 12-24 hours prior to the time of calculation. According to some embodiments, the calculation is a function of the evaporation time and evaporation rate of each one of plurality of heaters 164 in the 2-4, 4-12, 12-24 or 12-48 hours prior to the time of calculation. Each possibility represents a separate embodiment.

According to some embodiments, processing unit 204 comprises instructions to operate plurality of heaters 164, to enable a predetermined total amount of vaporized cannabinoids per period. According to some embodiments, the period is in the range of 4-8 hours. According to some embodiments, the period is in the range of 12-24 hours. According to some embodiments, the period is in the range of 24-48 hours. According to some embodiments, the predetermined total amount of vaporized cannabinoids is in the range of 0.5 to 10 milligrams. According to some embodiments, the predetermined total amount of vaporized cannabinoids is in the range of 1 to 5 milligrams. According to some embodiments, the predetermined total amount of vaporized cannabinoids is calculated by processing unit 204 as a function of the evaporation rate and the evaporation time.

Another optional parameter which controls the vaporization rate is the number of well(s) simultaneously operated, according to some embodiments. As detailed herein processing unit 204 is configured to operate each one of plurality of heaters 164 individually. Therefore, according to some embodiments, processing unit 204 is configured to operate a number of wells 152 simultaneously. Therefore, according to some embodiments, processing unit 204 is configured to operate at least 2, 3, 4 or 5 wells 152 simultaneously.

According to some embodiments, processing unit 204 is configured to simultaneously operate n of plurality of heaters 164, wherein n is an integer greater than 1. According to some embodiments, n is an integer greater than 2. According to some embodiments, n is an integer greater than 3. According to some embodiments, n is an integer greater than 4. According to some embodiments, n is an integer greater than 5. According to some embodiments, n is an integer greater than 6. According to some embodiments, n is an integer greater than 7. According to some embodiments, n is an integer greater than 8. According to some embodiments, n is an integer greater than 9. According to some embodiments, n is an integer greater than 10.

According to some embodiments, each of the n heaters 164 is configured to elevate the temperature of a corresponding well 152 containing cannabinoid concentrate 160 within its well cavity 158. According to some embodiments, upon the simultaneous operation of the n heaters 164 a cannabinoid vapor is formed at a rate substantially equal to n times the predetermined rate of a single well 152. According to some embodiments, the vaporization rate equals to n times the predetermined rate of a single well 152.

According to some embodiments, the predetermined temperature is in the range of 160° C. to 480° C.

According to some embodiments, the predetermined temperature is at least 160° C. According to some embodiments, the predetermined temperature is at least 180° C. According to some embodiments, the predetermined temperature is at least 200° C. According to some embodiments, the predetermined temperature is at least 250° C. According to some embodiments, the predetermined temperature is at least 300° C.

According to some embodiments, the predetermined temperature is no more than 480° C. According to some embodiments, the predetermined temperature is no more than 450° C. According to some embodiments, the predetermined temperature is no more than 425° C. According to some embodiments, the predetermined temperature is no more than 400° C.

According to some embodiments, the predetermined rate is in the range of 1 to 1000 micrograms per second.

According to some embodiments, the predetermined rate is at least 1 microgram per second. According to some embodiments, the predetermined rate is at least 2.5 micrograms per second. According to some embodiments, the predetermined rate is at least 5 micrograms per second. According to some embodiments, the predetermined rate is at least 10 micrograms per second. According to some embodiments, the predetermined rate is at least 15 micrograms per second. According to some embodiments, the predetermined rate is at least 25 micrograms per second. According to some embodiments, the predetermined rate is at least 40 micrograms per second. According to some embodiments, the predetermined rate is at least 50 micrograms per second. According to some embodiments, the predetermined rate is at least 75 micrograms per second. According to some embodiments, the predetermined rate is at least 100 micrograms per second. According to some embodiments, the predetermined rate is at least 150 micrograms per second. According to some embodiments, the predetermined rate is at least 200 micrograms per second. According to some embodiments, the predetermined rate is at least 250 micrograms per second.

According to some embodiments, n times the predetermined rate is at least 50 micrograms per second. According to some embodiments, n times the predetermined rate is at least 75 micrograms per second. According to some embodiments, n times the predetermined rate is at least 100 micrograms per second. According to some embodiments, n times the predetermined rate is at least 150 micrograms per second. According to some embodiments, n times the predetermined rate is at least 200 micrograms per second. According to some embodiments, n times the predetermined rate is at least 250 micrograms per second.

According to some embodiments, the predetermined rate is no more than 1000 micrograms per second. According to some embodiments, the predetermined rate is no more than 1000 micrograms per second. According to some embodiments, the predetermined rate is no more than 900 micrograms per second. According to some embodiments, the predetermined rate is no more than 750 micrograms per second. According to some embodiments, the predetermined rate is no more than 600 micrograms per second. According to some embodiments, the predetermined rate is no more than 500 micrograms per second. According to some embodiments, the predetermined rate is no more than 400 micrograms per second.

According to some embodiments, n times the predetermined rate is no more than 1000 micrograms per second. According to some embodiments, n times the predetermined rate is no more than 1000 micrograms per second. According to some embodiments, n times the predetermined rate is no more than 900 micrograms per second. According to some embodiments, n times the predetermined rate is no more than 750 micrograms per second. According to some embodiments, n times the predetermined rate is no more than 600 micrograms per second. According to some embodiments, n times the predetermined rate is no more than 500 micrograms per second. According to some embodiments, n times the predetermined rate is no more than 400 micrograms per second.

FIG. 3 is an exemplary operation of aerosol generating device 100. Specifically, in aerosol generating device 100 shown in FIG. 3, tray 151 includes five wells 152, numbered 152a-e. Of wells 152a-e, wells 152a-c and 152e include cannabinoid concentrate 160 in cavities 158 thereof, whereas well 152d is substantially empty. This state is achieved upon the consumption of cannabinoid concentrate 160 which was present originally in well 152d. For example, each well of wells 152a-e was originally provided with 5 milligrams of a cannabinoid concentrate 160. Also, the user of aerosol generating device 100 of this example is allowed to use 2.5 milligrams cannabinoid concentrates per day. As described herein, processing unit 204 is configured to control the periodically (e.g. daily) dosage of consumed cannabinoid concentrates, and in this example it was restricted to 2.5 milligrams cannabinoid concentrates per day, per physician's prescription. After one day of usage, half of the cannabinoid concentrate originally presented in well 152d was consumed and half remained (not shown in FIG. 3). After two days, further 2.5 milligrams cannabinoid concentrates were consumed from well 152d to reach the state depicted in FIG. 3.

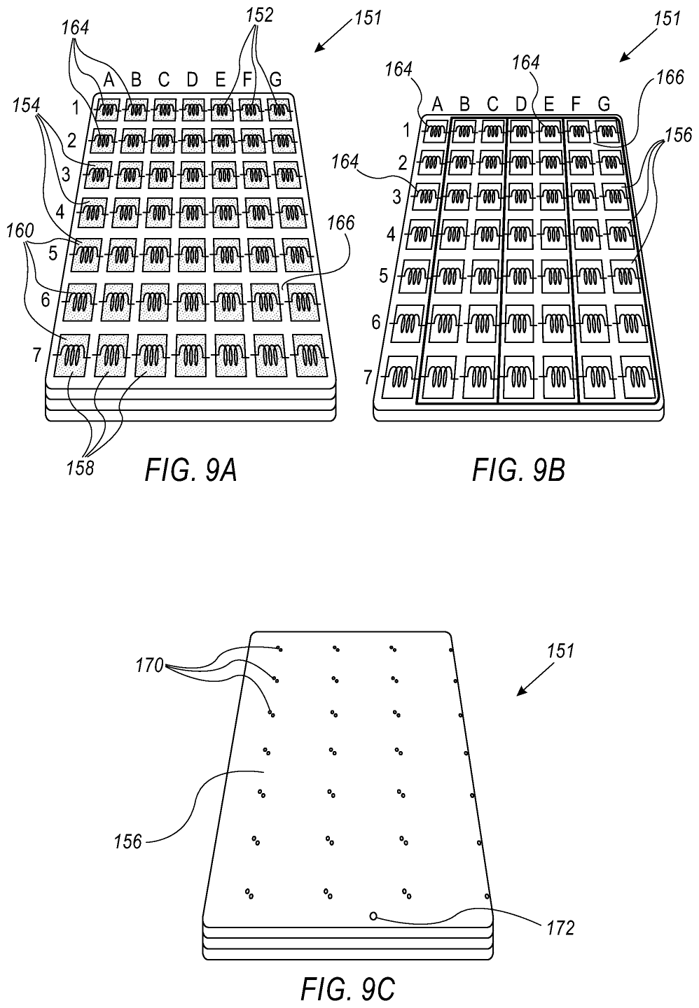

It is to be understood that the limitations of presentation size in some the figures (e.g. FIGS. 3-4) limit the number of wells 152 in the corresponding aerosol generating devices 100 to five (in FIG. 3) or six (FIG. 4). However, a larger number of wells may be used, e.g. by using a 2 dimensional well matrix, i.e. 6×6, 7×5, etc. as shown, e.g., in FIGS. 9A-C, 10A-C and 12A-C. Therefore, the total amount of cannabinoids is not restricted to small amounts.

Reference is now made to FIG. 4. FIG. 4 is an exemplary operation of aerosol generating device 100. Specifically, in aerosol generating device 100 shown in FIG. 4, tray 151 includes six wells 152, which are not numbered separately due to drawing constraints. Six wells 152 are referred as: first to the right well 152—closest to ground 210 upon assembly of aerosol generating device 100; second to the right well 152—adjacent to first to the right well 152; third to the right well 152—adjacent to second to the right well 152, but not to first to the right well 152; fourth to the right well 152—adjacent to third to the right well 152, but not to second to the right well 152; fifth to the right well 152—adjacent to fourth to the right well 152, but not to third to the right well 152; sixth to the right well 152—adjacent to fifth to the right well 152, but not to fourth to the right well 152. First to the right well 152 include full amount of cannabinoid concentrate 160. Second to the right well 152 include partial amount of cannabinoid concentrate 160. Fourth to the right well 152 include full amount of second composition 162. Fifth to the right well 152 include partial amount of second composition 162. Each one of wells third and sixth to the right 152 is substantially empty.

The state of aerosol generating device 100 depicted in FIG. 4 may be achieved as described in the following example. Cannabinoid concentrate 160 is THC enriched and second composition 162 is CBD enriched. Based on the concentrations of cannabinoids in the chemical compositions of cannabinoid concentrate 160 and second composition 162, a user wishes to consume 1:1 ratio of cannabinoid concentrate 160 to second composition 162. Before the initiation of cannabinoid consumption aerosol generating device 100 was provided with first, second and third to the right wells 152 each filled with 4 milligrams cannabinoid concentrate 160 and fourth-sixth to the right wells, each filled with 4 milligrams second composition 162. The aerosol generating device 100 user sets the ratio to 1:1 using the user interface as described below and to 4 milligrams per day total. On the first day the user operates aerosol generating device 100 through the user interface and starts inhaling. Then processing unit 204 operates simultaneously the third and sixth wells 152 to start consuming cannabinoid concentrate 160 and second composition 162 contained therein gradually—at the same vaporization rate. At some point, 2 milligram of cannabinoid concentrate 160 in the third well 152 and 2 milligram of second composition 162 in the sixth well are consumed and the operation is ceased for that day. At the end of the first day, wells sixth and third to the right 152 are half consumed. This operation is repeated for a second day. At the end of the second day, wells sixth and third to the right 152 are consumed and the other wells 152 are full. The operation is repeated once again for a third day. At the end of the third day, wells sixth and third to the right 152 are consumed and wells second and fifth to the right 152 are half consumed. This state is portrayed in FIG. 4.

Although this figure exemplifies a 1:1 ratio it is to be understood that upon proper instructions, ratio may be set to other values, such as 1:2, 2:3, 1:4 etc. according to some embodiments.

According to some embodiments, n of plurality of heaters 164 are each configured to elevate the temperature of a well 152 containing a cannabinoid concentrate 160 within its well cavity 158 and m of plurality of heaters 164 the plurality of heaters are each configured to elevate the temperature of a well 152 containing second composition 162 within its well cavity 158.

According to some embodiments, each of n and m is a an integer greater than zero. According to some embodiments, at least one of n and m is greater than one. According to some embodiments, n and m are equal. According to some embodiments, n and m are not equal.

According to some embodiments, processing unit 204 is configured to simultaneously operate each of the n and m of plurality of heaters 164. According to some embodiments, upon the simultaneous operation of the n and m of plurality of heaters 164 a vapor of cannabinoid concentrate 160 is formed at a rate substantially equal to n times the predetermined rate and a vapor of the second composition 162 is formed at a rate substantially equal to m times a second predetermined rate.

According to some embodiments, the predetermined rate is at least a half and not more than twice the second predetermined rate.

As aerosol generating device 100 is specifically configured to produce aerosols from viscous compositions, second composition 162 may be viscous, according to some embodiments. As aerosol generating device 100 is specifically configured to produce aerosols from cannabinoid compositions and is typically for cannabinoid users, second composition 162 may contain cannabinoids, such as concentrates, according to some embodiments.

According to some embodiments, second composition 162 has a viscosity of at least 1000 mPa·s. According to some embodiments, second composition 162 has a viscosity of at least 2000 mPa·s. According to some embodiments, second composition 162 has a viscosity of at least 3000 mPa·s. According to some embodiments, second composition 162 has a viscosity of at least 4000 mPa·s.

According to some embodiments, the second composition 162 is a second cannabinoid concentrate 162. According to some embodiments, one of cannabinoid concentrate 160 and second composition 162 is a THC enriched cannabinoid concentrate, and the other one of second composition 162 and cannabinoid concentrate 160 is a CBD enriched cannabinoid concentrate.

Back with reference to each one of FIGS. 1-6, and as detailed above, another feature of the present aerosol generating device 100 is that it may be controlled by a user or physician, through a user interface, according to some embodiments.

According to some embodiments, there is provided an aerosol generating system comprising aerosol generating device 100 and a user interface configured to send instruction signals to processing unit 204.

According to some embodiments, the user interface is embedded on the aerosol generating device 100. For example, the user interface may include a touch screen or a keyboard and screen embedded on controlling member housing 212. In such instances the user interface would be electrically connected to processing unit 204 to send (and optionally receive) electric instruction signals thereto.

According to some embodiments, the user interface is electrically wired to processing unit 204. According to some embodiments, the user interface is configured to send electric signals to processing unit 204. According to some embodiments, processing unit 204 is configured to send electric signals to the user interface. According to some embodiments, the electric signals may include any of the instructions detailed herein. According to some embodiments, at least some of the signals are corresponding to any one of the parameters specified herein.

According to some embodiments, the user interface is embedded on an external device. Such external devices may be, e.g. a smartphone, a tablet, a laptop or a desktop, having an application or software installed therein to communicate with processing unit 204. The external device may belong to the user and/or to a physician or other care-giver. Also, there may be a number of user interface, such as a physician computer, a user's smartphone and/or a touch screen on controlling member housing 212.

According to some embodiments, the user interface comprises a transmitter and the processing unit 204 comprises a receiver. According to some embodiments, the user interface is configured to send wireless signals to processing unit 204 through its transmitter, to be received by the receiver of processing unit 204. According to some embodiments, the wireless signals may include any of the instructions detailed herein. According to some embodiments, at least some of the signals are corresponding to any one of the parameters specified herein.

According to some embodiments, processing unit 204 comprises a transmitter and the user interface comprises a receiver, wherein processing unit 204 is configured to send wireless signals to the user interface through its transmitter, to be received by the user interface receiver

According to some embodiments, the user interface is configured to send instruction signals to processing unit 204 to effect at least one parameter selected from the group consisting of: n, the predetermined rate and the predetermined temperature. Each possibility represents a separate embodiment. According to some embodiments, the parameter is n. According to some embodiments, the parameter is the predetermined rate. According to some embodiments, the parameter is the predetermined temperature. According to some embodiments, a combination of parameters are affected.

According to some embodiments, the instruction signals effect at least one parameter selected from the group consisting of: n and the predetermined temperature, thereby effecting the predetermined rate. Each possibility represents a separate embodiment.

According to some embodiments, the user interface is configured to send instruction signals to processing unit 204 to effect the predetermined rate. According to some embodiments, the user interface is further configured to send instruction signals to the processing unit 204 to effect a duration of the operation of the heaters, thereby controlling the total amount of cannabinoid concentrate being evaporated.

According to some embodiments, the user interface is controllable by a user, and at least one of the parameters is controllable by the user.

According to some embodiments, the user interface requires a permit, and control of the total amount of cannabinoid concentrate being evaporated is controllable by a holder of the permit. According to some embodiments, the holder of the permit is a physician.

According to some embodiments, wherein the user interface is further configured to send instruction signals to the processing unit to effect at least one parameter selected from the group consisting of: m and the second predetermined rate.

According to some embodiments, the processing unit is configured to calculate the amount of cannabinoid concentrate evaporated, to record results of said calculation, and to send wireless recordation signals to the user interface, wherein the wireless recordation signals are indicative of said recording.

According to some embodiments, the calculation is based on at least one parameter selected from the group consisting of n, the predetermined rate and the predetermined temperature. According to some embodiments, the calculation is based on at least the evaporation rate. According to some embodiments, the calculation is based on at least the evaporation time. According to some embodiments, the evaporation rate is calculated by processing unit 204 based on at least one parameter selected from: the composition of cannabinoid concentrate 160, the evaporation temperature and n. According to some embodiments, the evaporation rate is calculated by processing unit 204 based on at least one parameter selected from: the composition of cannabinoid concentrate 160, and the evaporation temperature.

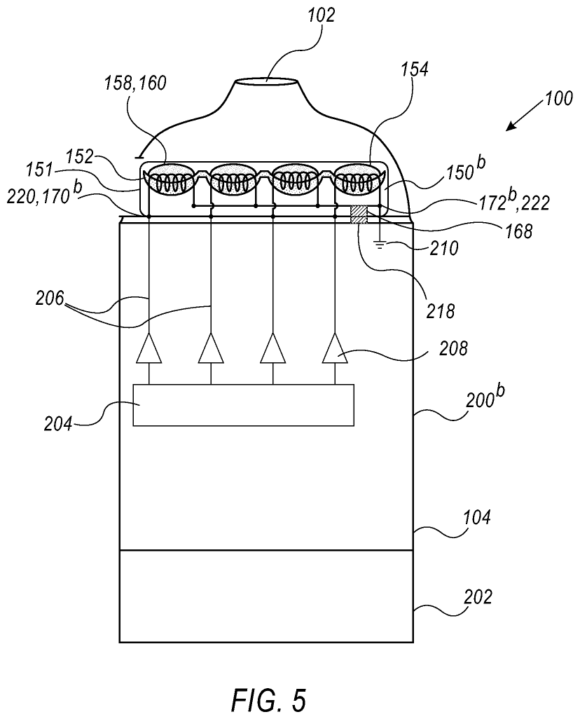

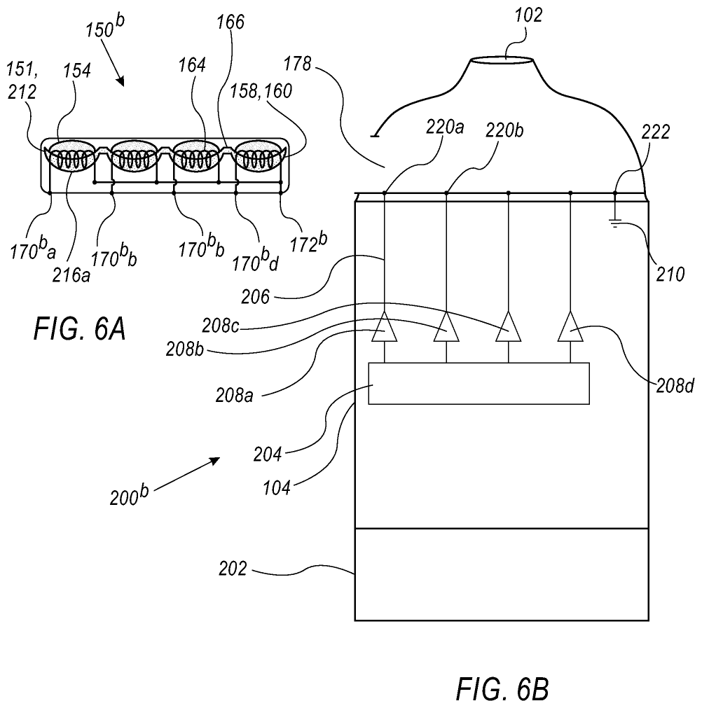

Reference is now made to FIGS. 5-6 which illustrate a different setup of aerosol generating device 100 than the one described in FIGS. 1-4. Specifically, in FIGS. 1-4, aerosol generating device 100 is divided to cartridge 150^{a}, intended to be disposable and durable controlling member 200^{a}. In FIGS. 5-6, the disposables/consumables are assembled in an insertable/removable cassette 150^{b}. The remaining parts, including mouthpiece 106 and outlet 102, are assembled within an aerosol generating device housing 104 of a controlling member 200^{b}. Controlling member 200^{b}, his aerosol generating device housing 104 and the components included therein are intended to be durable. According to some embodiments, cassette 150^{b} is intended for use only until the cannabinoid concentrate 160 contained in its plurality of wells 152 is consumed, whereas controlling member 200^{b} is durable and after consumption of the cannabinoid concentrate 160 contained in a first cassette 150^{b}, a second cassette 150^{b} may be mounted/assembled on controlling member 200^{b} for a further sequence of aerosolizations.

As with aerosol generating device 100 of FIGS. 1-4, aerosol generating device 100 of FIGS. 5-6 is considered assembled upon insertion of cassette 150^{b} into controlling member 200^{b}, and disassembled upon its removal. As Seen in FIGS. 5-6, aerosol generating device housing 104 includes a slot 178, according to some embodiments. Slot 178 is configured to enable insertion of cassette 150^{b} into controlling member 200^{b} through aerosol generating device housing 104 and to remove it, according to some embodiments.

Also as with cartridge 150^{a} of aerosol generating device 100 of FIGS. 1-4, cassette 150^{b} may include plurality of cassette electric contacts 170^{b}, each configured to make electric contact with a corresponding one of plurality of controlling member contacts 220 upon assembly of aerosol generating device 100, according to some embodiments. Finally, according to some embodiments, cassette 150^{b} may comprise cassette ground contact 172^{b} to be connected to ground 210. The mechanism is similar to and further elaborated when discussing ground 210 of aerosol generating device 100 of FIGS. 1-4 above.

It is to be understood that, while the assembly specifications of aerosol generating device 100 of FIGS. 5-6 are distinct from the assembly specifications of aerosol generating device 100 of FIGS. 1-4, other features of aerosol generating device 100 are similar. Such features include, inter alia, the operation, control, user interface, processing unit 204, plurality of wells 152, tray 151, outlet 102, plurality of heaters 164.

According to some embodiments, aerosol generating device 100 comprises:
aerosol generating device housing 104, which houses processing unit 204, mouthpiece 106 comprising outlet 102 and a power source compartment; and
a cassette 150^{b} comprising tray 151 and plurality of heaters 164.

According to some embodiments, the aerosol generating device housing 104 comprises inlet slot 178 configured for insertion of cassette 150^{b} into the aerosol generating device housing 104. According to some embodiments, upon insertion of cassette 150^{b} into inlet slot 178, aerosol generating device 100 is assembled. According to some embodiments, upon removal of cassette 150^{b} through inlet slot 178 from aerosol generating device housing 104, aerosol generating device 100 is disassembled. According to some embodiments, upon assembly of aerosol generating device 100, processing unit 204 forms an electric contact with each one of plurality of heaters 164 separately.

According to some embodiments, the aerosol generating device further comprises reader 218 housed within aerosol generating device housing 104. According to some embodiments, the cassette 150^{b} comprises an identifier 168 at an external surface of distal face thereof. According to some embodiments, upon assembly of aerosol generating device 100, identifier 168 is positioned to face reader 218. According to some embodiments, reader 218 is configured to identify identifier 168 and to send identification signals indicative of the identification to processing unit 204. According to some embodiments, the identification comprises information about contents within each well cavity 158 of the plurality of wells 152. According to some embodiments, the identifier 168 is a barcode and reader 218 is a barcode reader. According to some embodiments, the identifier 168 is a QR code and reader 218 is a QR code reader.

FIG. 7 is an exemplary operation of aerosol generating device 100, which comprises: aerosol generating device housing 104, which houses processing unit 204, mouthpiece 106 comprising outlet 102 and a power source compartment; and a cassette 150^{b} comprising tray 151 and plurality of heaters 164. Specifically, in aerosol generating device 100 shown in FIG. 7, tray 151 includes five wells 152. Like wells 152 of FIG. 3 (wells 152a-e), four wells 152 include cannabinoid concentrate 160 in cavities 158 thereof, whereas one well is substantially empty. This state is achieved upon the consumption of cannabinoid concentrate 160 which was present originally in the empty well. For example, each well of wells 152 was originally provided with 4 milligrams of a cannabinoid concentrate 160. Also, the user of aerosol generating device 100 of this example is allowed to use 1 milligrams cannabinoid concentrates per 6 hours. As described herein, processing unit 204 is configured to control the periodically (e.g. every 6 hours) dosage of consumed cannabinoid concentrates, and in this example it was restricted to 1 milligrams cannabinoid concentrates per 6 hours, per physician's prescription. After the first 6 hours of usage, a quarter of the cannabinoid concentrate originally presented in well 152 was consumed and ¾ remained (not shown in FIG. 7). After the next 6 hours of usage, a half of the cannabinoid concentrate originally presented in well 152 was consumed and ½ remained (not shown in FIG. 7). After the next 6 hours of usage (18 hours since beginning), ¾ of the cannabinoid concentrate originally presented in well 152 was consumed and ¼ remained (not shown in FIG. 7). After the fourth 6 hour period, further 1 milligram cannabinoid concentrates was consumed from well 152 to reach the empty state depicted in FIG. 7.

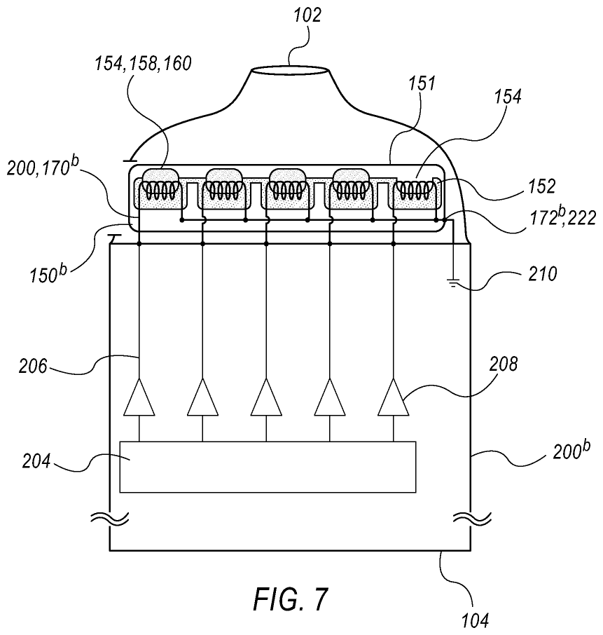

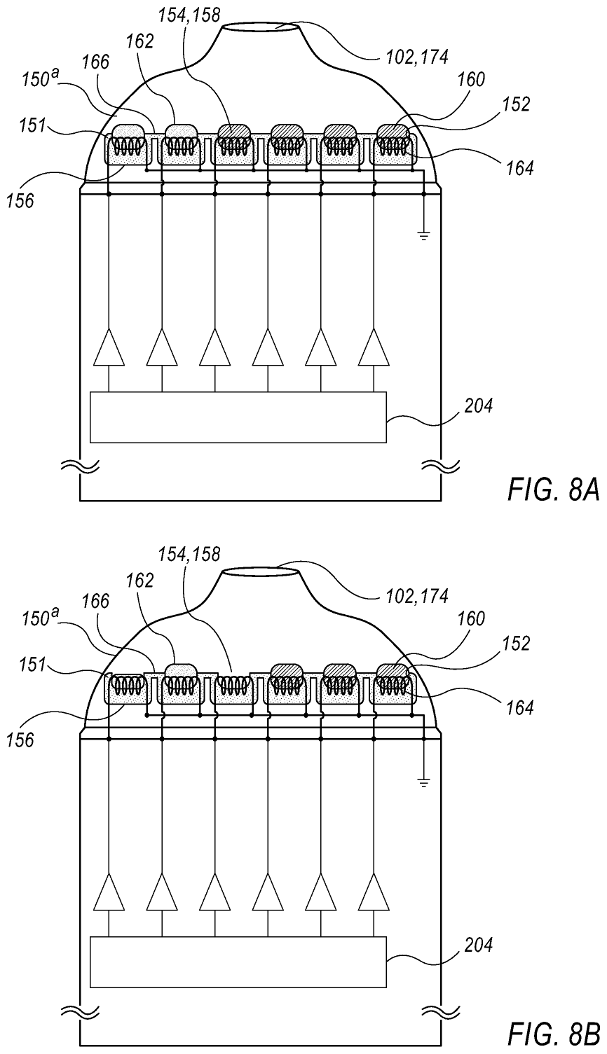

It is to be understood that the limitations of presentation size in some the figures (e.g. FIGS. 7-8) limit the number of wells 152 in the corresponding aerosol generating devices 100 to five (in FIG. 7) or six (FIG. 8). However, a larger number of wells may be used, e.g. by using a 2 dimensional well matrix, i.e. 6×6, 7×5, etc. as shown, e.g., in FIGS. 9A-C, 10A-C and 12A-C. Therefore, the total amount of cannabinoids is not restricted to small amounts.

FIGS. 8A and 8B represent an exemplary operation of aerosol generating device 100, which comprises cartridge 150^{a} and a controlling member 200^{a}, as described herein, according to some embodiments. Specifically, in aerosol generating device 100 shown in FIGS. 8A-B, tray 151 includes six wells 152, which are not numbered separately due to drawing constraints. Six wells 152 are referred as: first to the right well 152—closest to ground 210 upon assembly of aerosol generating device 100; second to the right well 152—adjacent to first to the right well 152; third to the right well 152—adjacent to second to the right well 152, but not to first to the right well 152; fourth to the right well 152—adjacent to third to the right well 152, but not to second to the right well 152; fifth to the right well 152—adjacent to fourth to the right well 152, but not to third to the right well 152; sixth to the right well 152—adjacent to fifth to the right well 152, but not to fourth to the right well 152.

In FIG. 8A, fifth to the right and sixth to the right wells 152, each includes a full amount of second composition 162 and first, second, third and fourth to the right each includes a full amount of cannabinoid concentrate 160. According to some embodiments, aerosol generating device 100 is provided initially wherein each of plurality of wells 152 is full with either cannabinoid concentrate 160 or second composition 162. In FIG. 8B, first to the right, second to the right and third to the right wells 152, each includes a full amount of cannabinoid concentrate 160; fourth to the right well 152 is empty, fifth to the right well 152 includes a full amount of second composition 162 and sixth to the right well 152 includes a partial amount of second composition 162.

Specifically, aerosol generating device 100 shown in FIG. 8A has a disposable cartridge 150^{a}. Said cartridge is provided with 2:1 ratio between wells 152 having cannabinoid concentrate 160 (four wells 152) and wells 152 having second composition 162 (two wells 152), according to some embodiments. In the present example, cannabinoid concentrate 160 is a THC enriched concentrate and second composition 162 is a CBD enriched concentrate. Therefore, the total THC enriched composition amount in cartridge 150^{a} of FIG. 8A is twice than the CBD enriched composition amount therein. Such cartridge is thus suitable for a user, who wants to inhale THC/CBD aerosol at about a specific ratio, which is the result of the concentration of THC and CBD in cannabinoid concentrate 160 and second composition 162.

The state of tray 151 depicted in FIG. 8B may be achieved as described in the following example. Cannabinoid concentrate 160 is THC enriched and second composition 162 is CBD enriched. Based on the concentrations of cannabinoids in the chemical compositions of cannabinoid concentrate 160 and second composition 162, a user wishes to consume 2:1 ratio of cannabinoid concentrate 160 to second composition 162. Thus, a cannabinoid consumer decided to purchase cartridge 150^{a} having this desired compositional ratio.

Before the initiation of cannabinoid consumption aerosol generating device 100 was provided as shown in FIG. 8A with first, second, third and fourth to the right wells 152 each filled with 10 milligrams cannabinoid concentrate 160 and fifth and sixth to the right wells 152, each filled with 10 milligrams second composition 162. The aerosol generating device 100 user after purchasing the cartridge 150^{a} having the desired composition, is assembling aerosol generating device 100 by connecting between cartridge 150^{a} and controlling member 200^{a}. identifier 168 is then being automatically read by reader 218, such that processing unit 204 receives an indication of the compositional contents in each of six wells 152.

Thereafter, the aerosol generating device 100 user sets the ratio to 2:1 cannabinoid concentrate 160 to second composition 162 using the user interface as described herein and to 7.5 milligrams per day total. On the first day the user operates aerosol generating device 100 through the user interface and starts inhaling. Then processing unit 204 operates simultaneously the fourth to the right and sixth to the right wells 152 to start heating and aerosolizing cannabinoid concentrate 160 and second composition 162 contained therein gradually. Since the ratio was set to 2:1 by the user, the vaporization rate at the fourth to the right well 152 is twice than the vaporization rate at the sixth to the right well 152. At some point during operation, 5 milligram of cannabinoid concentrate 160 in the third well 152 and 2.5 milligram of second composition 162 in the sixth well are consumed and the operation is ceased for that day. At the end of the first day, well fourth to the right 152 is half consumed, and well sixth to the right 152 is ¼ consumed (with 7.5 of 10 milligrams of the second composition 162 remaining). This operation is repeated for a second day. At the end of the second day, well fourth to the right 152 is consumed; well six to the right 152 is consumed; wells first, second and third to the right 152 are each full with 10 milligrams of cannabinoid concentrate 160; and well fifth to the right 152 is full with 10 milligrams of second composition 162. This state is portrayed in FIG. 8B.

Although this figure exemplifies a 1:2 ratio it is to be understood that upon proper instructions, ratio may be set to other values, such as 1:1, 2:3, 1:4 etc. according to some embodiments.

Reference is now made to FIGS. 9A-C. FIGS. 9A-C are depicting tray 151 as described above, separated from aerosol generating device 100, from different view. Specifically, FIG. 9A is top view of tray 151. By “top view” it is meant that the point of inspection is from the proximal side, i.e. when aerosol generating device 100 is assembled with tray 151 therein, the point of view is from the outlet 102 towards tray 151. FIG. 9B is top cross sectional view of tray 151, which enables a look into a distal section thereof. FIG. 9C is bottom view of tray 151. By “bottom view” it is meant that the point of inspection is from the distal side, i.e. when aerosol generating device 100 is assembled with tray 151 therein, the point of view is from processing unit 204 towards tray 151.

As detailed herein tray 151 comprises plurality of wells 152, each having open side 154, closed face 156 and well cavity 158, according to some embodiments. Tray 151 further comprises inter-well joints 166 between plurality of wells 152 and plurality of heaters 164, each corresponding to one of plurality of wells 152. In tray 151 shown in FIGS. 9A-C, each of plurality of wells 152 contains a cannabinoid concentrate 160.

The separate portrayal of tray 151 in FIGS. 9A-C enables to present a two-dimensional embodiment of tray 151, which is mentioned above. According to some embodiments, tray 151 comprises plurality of wells 152 arranged in a two dimensional matrix having at least two vertical tray 151 rows and at least two horizontal tray 151 rows. According to some embodiments, the matrix has at least three vertical tray 151 rows. According to some embodiments, the matrix has at least 4 vertical tray 151 rows. According to some embodiments, the matrix has at least 5 vertical tray 151 rows. According to some embodiments, the matrix has at least 6 vertical tray 151 rows. According to some embodiments, the matrix has at least 7 vertical tray 151 rows. According to some embodiments, the matrix has at least 8 vertical tray 151 rows. According to some embodiments, the matrix has at least 9 vertical tray 151 rows. According to some embodiments, the matrix has at least 10 vertical tray 151 rows. According to some embodiments, the matrix has at least three horizontal tray 151 rows. According to some embodiments, the matrix has at least 4 horizontal tray 151 rows. According to some embodiments, the matrix has at least 5 horizontal tray 151 rows. According to some embodiments, the matrix has at least 6 horizontal tray 151 rows. According to some embodiments, the matrix has at least 7 horizontal tray 151 rows. According to some embodiments, the matrix has at least 8 horizontal tray 151 rows. According to some embodiments, the matrix has at least 9 horizontal tray 151 rows. According to some embodiments, the matrix has at least 10 horizontal tray 151 rows.

In FIGS. 9A-B the horizontal well 152 rows are numbered 1-7 and the vertical well 152 rows are numbered A-G, such that each well 152 can receive an individual reference number.

In FIG. 9A, cannabinoid concentrate 160 is presented as half transparent, such that plurality of heaters 164 can be seen therethrough, partially inside plurality of wells 152. In FIG. 9B, the bottom layer of tray 151 is portrayed. Since only a bottom part of tray 151 is shown in FIG. 9B, a corresponding bottom part of each one of plurality of wells 152 is shown. Thus, that closed face 156 is visible, whereas open side 154 is not. Accordingly, cannabinoid concentrate 160 and well cavity 158 are not indicated. In FIG. 9C tray 151 is shown from below, such that plurality of cartridge electric contacts 170, closed face 156 and ground contact 172 (e.g. cartridge ground contact 172^{a} or cassette ground contact 172^{b}) are visible.

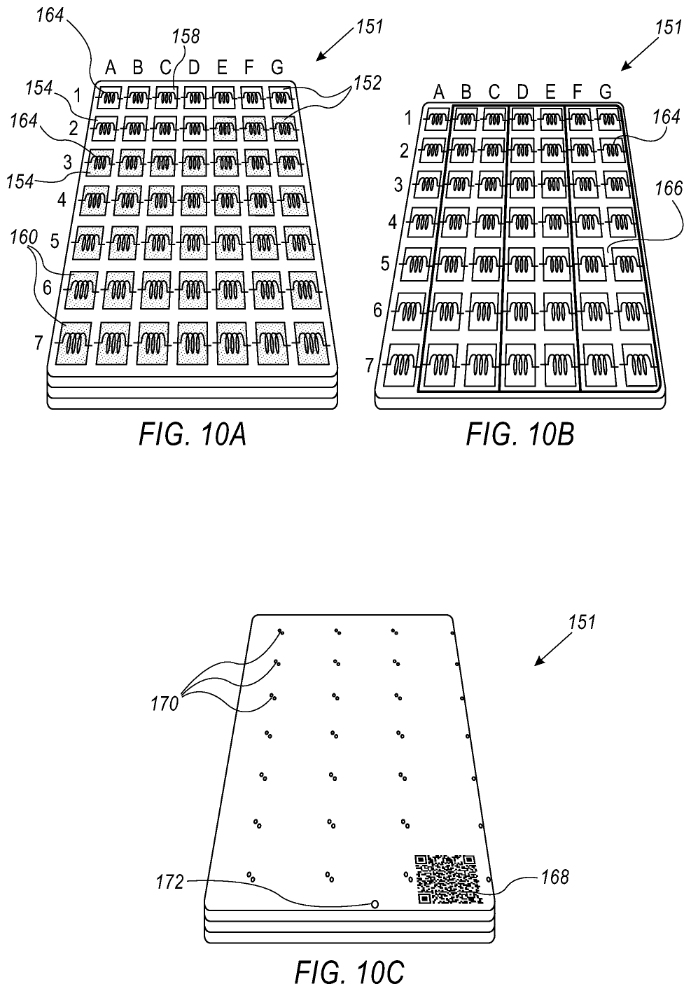

Reference is now made to FIGS. 10A-C. FIGS. 10A-C depict a tray 151 similar to that shown in FIGS. 9A-C. In tray 151 of FIGS. 10A-C identifier 168 is specifically represented as a QR code. In addition, tray 151 of FIGS. 10A-C includes a matrix of wells 152, comprising 7 well 152 horizontal rows and 7 well 152 vertical rows. Horizontal well 152 rows are numbered 1-7 and the vertical well 152 rows are numbered A-G, such that each well 152 can receive an individual reference number. In tray 151 of FIGS. 10A-C, 11 of plurality of wells 152—wells 1A-G and 2A-D are empty and the other 38 wells 152 are filled with cannabinoid concentrate 160, as shown in FIG. 10A, according to some embodiments.

Specifically, FIGS. 10A-C represent states of tray 151 in an exemplary operation of aerosol generating device 100 (whether as represented in FIGS. 1,2, 3, 4 and 8 with cartridge 150^{a} or as represented in FIGS. 5, 6 and 7 with cassette 150^{b}). Specifically, tray 151 shown in FIG. 7 includes 49 wells 152. Out of 49 wells 152 of tray 151, 38 wells 52 include cannabinoid concentrate 160 in cavities 158 thereof, whereas 11 wells 152 are substantially empty. This state is achieved upon the consumption of cannabinoid concentrate 160 which was present originally in the empty wells 152. For example, each one of the 49 wells 152 was originally provided with 2 milligrams of a cannabinoid concentrate 160 (e.g. as shown in FIG. 9A). Also, the user of aerosol generating device 100 of this example is allowed to use 1 milligrams cannabinoid concentrates per day. As described herein, processing unit 204 is configured to control the periodically (e.g. every day) dosage of consumed cannabinoid concentrates, and in this example it was restricted to 2 milligrams cannabinoid concentrates per day, per physician's prescription. After the day of usage, the cannabinoid concentrate 160 originally presented in well 1A (wells are generally numbered as element 152, but for convenience, the specific well designation is used for the remainder of the example) was consumed and the cannabinoid concentrate 160 in the other wells (1B-G and 2-7A-G) still remains in the respective well cavities 158. After the second day of usage, well 1B is also consumed and so on. After the 11^{th} day, each one of wells 1A-G and 2A-D is empty after consumption by the user and the other wells (2D-G and 3-7A-G) are still containing cannabinoid concentrate 160 within their cavities 158 to reach the state depicted in FIG. 10A.

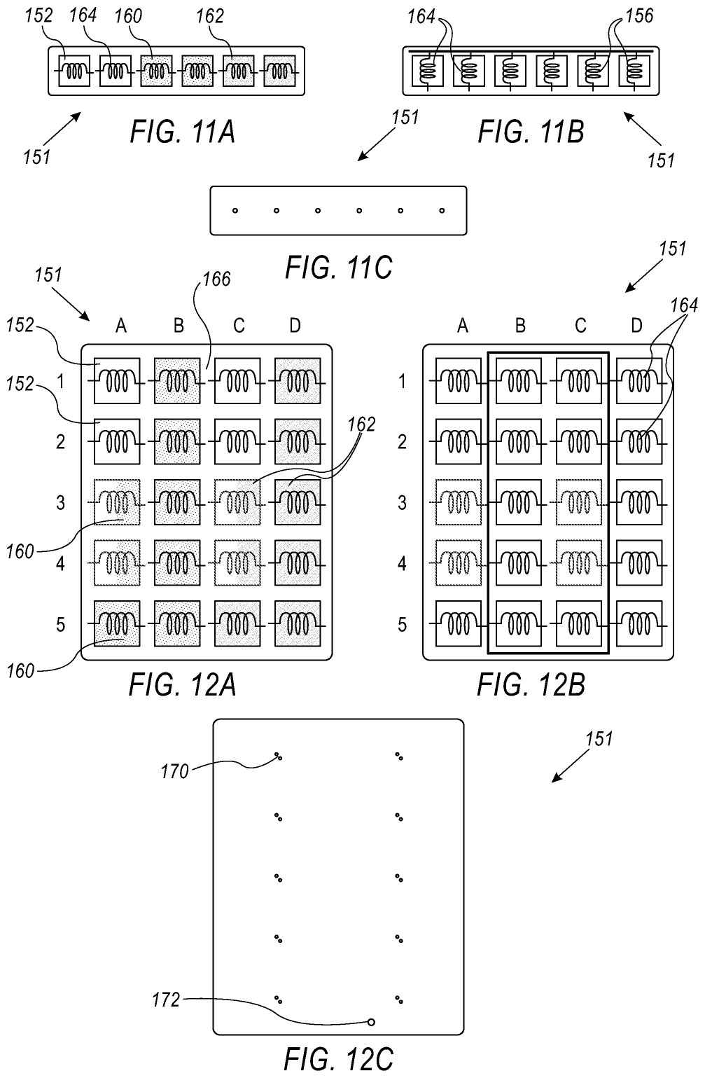

FIGS. 11A-C show a 1 dimensional tray from a top (FIG. 11A) top cross sectional (FIG. 11B) and bottom (FIG. 11C) views. FIGS. 11A-C represent tray 151 during an exemplary operation of aerosol generating device 100. Specifically, tray 151 of FIGS. 11A-C includes six wells 152, which are not numbered separately due to drawing constraints. Six wells 152 are referred as: first to the right well 152 to sixth to the right as described when referring to FIGS. 8A-B.

Before the beginning of aerosolizations from tray 151 of FIG. 11A-C, third, fourth, fifth and sixth to the right wells 152, each includes a full amount of cannabinoid concentrate 160; and first and second to the right wells 152 each includes a full amount of second composition 162. According to some embodiments, aerosol generating device 100 is provided initially wherein each of plurality of wells 152 is full with either cannabinoid concentrate 160 or second composition 162. In FIG. 11A, first to the right and second to the right wells 152, each still includes a full amount of second composition 162; third and fourth to the right wells 152 each still includes a full amount of cannabinoid concentrate 160; and fifth and sixth to the right wells 152 each is empty.

Specifically, tray 151 is originally provided with 2:1 ratio between wells 152 having cannabinoid concentrate 160 (four wells 152) and wells 152 having second composition 162 (two wells 152), according to some embodiments. In the present example, cannabinoid concentrate 160 is a THC enriched concentrate and second composition 162 is a CBD enriched concentrate. Therefore, the total THC enriched composition amount in tray 151 of FIGS. 11A-C is twice than the CBD enriched composition amount therein.

The state of tray 151 depicted in FIG. 11A may be achieved as described in the following example. Cannabinoid concentrate 160 is THC enriched and second composition 162 is CBD enriched. A user wishes to consume cannabinoid concentrate 160 only in the first four days of use.

Before the initiation of cannabinoid consumption tray 151 was provided with first and second to the right wells 152 each filled with 6 milligrams second composition 162; and third, fourth fifth and sixth to the right wells 152 each filled with 6 milligrams cannabinoid concentrate 160. The aerosol generating device 100 user after purchasing the cassette 150^{b} or cartridge 150^{a}, is assembling aerosol generating device 100. identifier 168 is then being automatically read by reader 218, such that processing unit 204 receives an indication of the compositional contents in each of six wells 152.

Thereafter, the aerosol generating device 100 user sets the ratio to 100% cannabinoid concentrate 160 aerosolization using the user interface as described herein and to 3 milligrams per day total. The user further sets the vaporization rate to ‘high’ using the user interface, as the user wishes to consume concentrated amount of cannabinoids at a short time period. In the following example, the ‘high’ rate is 1 milligram cannabinoid concentrate 160 vaporized in 5 seconds. On the first day the user operates aerosol generating device 100 through the user interface and starts inhaling. Since high dosage of cannabinoid concentrate 160 is to be evaporated at short time period, processing unit 204 operates simultaneously two heaters 164 corresponding to two wells 152, each containing cannabinoid concentrate 160—well fifth and sixth to the right 152. This action initiates heating and aerosolizing cannabinoid concentrate 160 contained therein at a rate of 0.2 milligrams per second. Since the daily dose was restricted to 3 milligrams, after 15 seconds, the processing unit 204 ceases the operation of the heaters and postpones operation until the next day. At this point, 1.5 milligram of cannabinoid concentrate 160 in the fifth to the right well 152 and 1.5 milligram of cannabinoid concentrate 160 in the sixth to the right well 152 are consumed and the operation is ceased for that day. At the end of the first day, each one of wells 152 fifth and sixth to the right are ¾ full and the other wells 152 are full with cannabinoid concentrate 160. This operation is repeated for a second day. At the end of the second day, each one of wells 152 fifth and sixth to the right are ½ full and the other wells 152 are full with cannabinoid concentrate 160. This operation is repeated for a third day. At the end of the second day, each one of wells 152 fifth and sixth to the right are ¼ full and the other wells 152 are full with cannabinoid concentrate 160. This operation is repeated for a fourth day, after which each one of wells 152 fifth and sixth to the right are empty and the other wells 152 are full with cannabinoid concentrate 160. This state is shown in FIG. 11A.

Reference is now made to FIGS. 12A-C, which again present a two-dimensional embodiment of tray 151. Tray 151 of FIGS. 12A-C includes a matrix of wells 152, comprising 5 well 152 horizontal rows and 4 well 152 vertical rows. Horizontal well 152 rows are numbered 1-5 and the vertical well 152 rows are numbered A-D, such that each well 152 can receive an individual reference number. Wells are generally numbered as element 152, but for convenience, the specific well designation is used for the remainder of the example. In tray 151 of FIGS. 12A-C, four of plurality of wells 152 (wells 1A, 2A, 1C and 2C) are empty; two of plurality of wells 152 (wells 3A, and 4A) are partially filled with cannabinoid concentrate 160; two of plurality of wells 152 (wells 3C, and 4C) are partially filled with second composition 162; six of plurality of wells 152 (wells 5A, and 1-5B) are filled with cannabinoid concentrate 160; and six of plurality of wells 152 (wells 5C, and 1-5D) are filled with second composition 162, as shown in FIG. 12A, according to some embodiments.

Before the beginning of aerosolizations from tray 151 of FIG. 12A-C, wells 152 in the A and B vertical rows, each includes a full amount of cannabinoid concentrate 160; and wells 152 in the C and D vertical rows each includes a full amount of second composition 162. According to some embodiments, aerosol generating device 100 is provided initially wherein each of plurality of wells 152 is full with either cannabinoid concentrate 160 and/or second composition 162. In FIG. 12A, wells 5C and 1-5D, each still includes a full amount of second composition 162; wells 5A and 1-5B each still includes a full amount of cannabinoid concentrate 160; wells 3-4C each still includes a half amount of second composition 162; wells 5A and 1-5B each still includes a full amount of cannabinoid concentrate 160; wells 3-4A each still includes a half amount of cannabinoid concentrate 160; and wells 1-2A and 1-2C each is empty.

Specifically, tray 151 is originally provided with 1:1 ratio between wells 152 having cannabinoid concentrate 160 (ten wells in vertical rows A and B) and wells 152 having second composition 162 (ten wells in vertical rows C and D), according to some embodiments. In the present example, cannabinoid concentrate 160 is a THC enriched concentrate and second composition 162 is a CBD enriched concentrate. Therefore, the total THC enriched composition amount in tray 151 of FIGS. 12A-C is substantially equal to the CBD enriched composition amount therein.

The state of tray 151 depicted in FIG. 12A may be achieved as described in the following example. Cannabinoid concentrate 160 is THC enriched and second composition 162 is CBD enriched. A user wishes to consume 1:1 ratio of cannabinoid concentrate 160 to second composition 162. Thus, the cannabinoid consumer decides to purchase cartridge 150^{a} or cassette 150^{b} having this desired compositional ratio.

Before the initiation of cannabinoid consumption tray 151 was provided with wells in vertical rows C and D each filled with 4 milligrams second composition 162; and wells in vertical rows A and B each filled with 4 milligrams of cannabinoid concentrate 160. The aerosol generating device 100 user after purchasing the cassette 150^{b} or cartridge 150^{a} having the desired composition, is assembling aerosol generating device 100. identifier 168 (not shown in FIGS. 12A-C) is then being automatically read by reader 218, such that processing unit 204 receives an indication of the compositional contents in each of 20 wells 152.

Thereafter, the aerosol generating device 100 user sets the ratio to 50% cannabinoid concentrate 160 and 50% second composition 162 aerosolization using the user interface as described herein and to 4 milligrams per day total. The user further sets the vaporization rate to ‘high’ using the user interface, as the user wishes to consume concentrated amount of cannabinoids at a short time period. In the following example, the ‘high’ rate is 1 milligram cannabinoids vaporized in 2.5 seconds. On the first day the user operates aerosol generating device 100 through the user interface and starts inhaling. Since high dosage of cannabinoid concentrate 160 is to be evaporated at short time period, processing unit 204 operates simultaneously four heaters 164 corresponding to wells 1A, 2A, 1C and 2C. This action initiates heating and aerosolizing cannabinoid concentrate 160 contained therein at a rate of 0.2 milligrams per second and second composition 162 at a rate of 0.2 milligrams per second. In total the rate is 0.4 mg cannabinoids per second, or 1 milligram cannabinoids per 2.5 seconds as set by the user. Since the total cannabinoid daily dose was restricted to 4 milligrams, after 10 seconds, the processing unit 204 ceases the operation of the heaters and postpones operation until the next day. At this point, 1 milligram of cannabinoid concentrate 160 each one of wells 1A and 2A is consumed and 1 milligram of cannabinoid concentrate 160 each one of wells 1C and 2C is consumed, and the operation is ceased for that day. At the end of the first day, each one of wells 1A and 2A contains 3 milligrams of the original 4 milligrams of cannabinoid concentrate 160; and each one of wells 1C and 2C contains 3 milligrams of the original 4 milligrams of second composition 162. This operation is repeated for three more days, after which each one of wells 1A, 2A, 1C and 2C is empty and the other wells are filled with either cannabinoid concentrate 160 or second composition 162. The operation is repeated for two more days, after which each one of wells 1A, 2A, 1C and 2C is empty; each one of wells 3A and 4A contains 2 milligrams of the original 4 milligrams of cannabinoid concentrate 160; each one of wells 4C and 4C contains 2 milligrams of the original 4 milligrams of second composition 162; and the other wells are filled with either cannabinoid concentrate 160 or second composition 162. This state is shown in FIG. 11A.

Lastly, the present disclosure provides a process to insert cannabinoid concentrate 160 and/or second composition 162 into well cavities 158 of plurality of wells 152.

According to some embodiments, wells 152 containing cannabinoid concentrate 160 within their well cavity 158 are produced by insertion of cannabinoid concentrate 160 concentrate into the well cavities 158. According to some embodiments, the insertion is performed by a procedure selected from:
placing undissolved cannabinoid concentrates over tray 151 and depositing the concentrates into well cavities 158 using a doctor blade; and
depositing dissolved cannabinoid concentrates in well cavities 158 and evaporating the solvent, optionally a plurality of times.

According to some embodiments, the insertion is performed by placing undissolved cannabinoid concentrates over tray 151 and depositing the concentrates into well cavities 158 using a doctor blade. According to some embodiments, the insertion is performed by depositing dissolved cannabinoid concentrates in the cavities 158 and evaporating the solvent, optionally a plurality of times. According to some embodiments, the insertion is performed by depositing dissolved cannabinoid concentrates in the cavities 158 and evaporating the solvent a plurality of times. According to some embodiments, the solvent is ethanol.

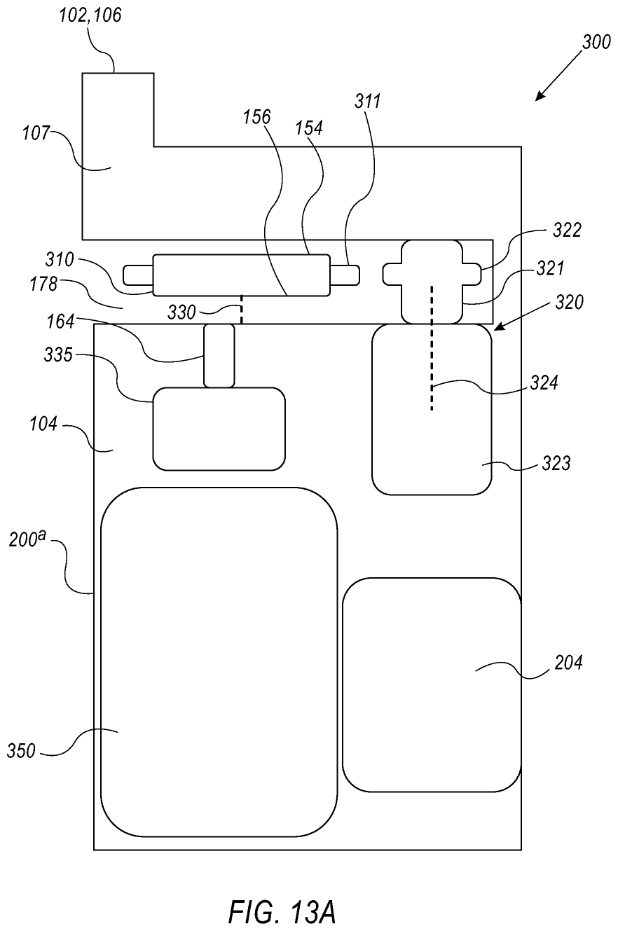

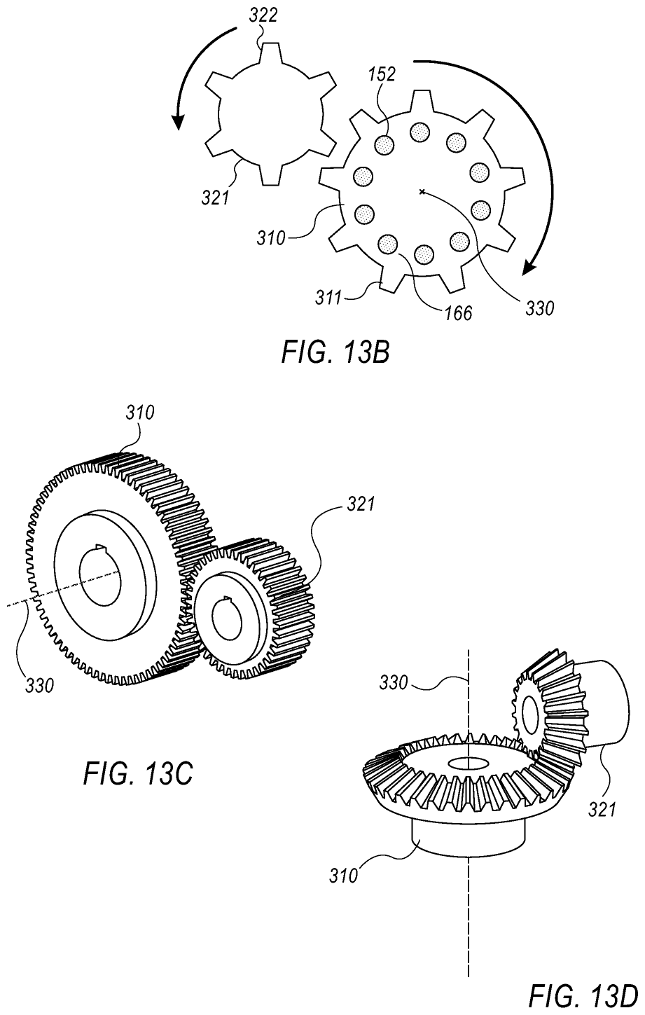

FIGS. 13A-13E constitute various views of various parts of an aerosol generating device 300, according to some embodiments. Particularly, FIG. 13A constitutes a cross-sectional view of aerosol generating device 300. FIG. 13B constitutes a top view of a portion of a first embodiment aerosol generating device 300. FIGS. 13C-13D constitute perspective views of various gear configurations. FIG. 13E constitutes a top view of a portion of a second embodiment of aerosol generating device 300. Aerosol generating device 300 comprises: a rotatable tray 310; a rotatable tray actuator 320; at least one heater 164; a processing unit 204; and an outlet 102. Rotatable tray 310 comprises at least one well 152 and/or 352, as will be described below. According to some embodiments, rotatable tray 310 exhibits a rotational axis 330 extending there through.

According to some embodiments, as described above, each well 152 has an open side 154, a closed face 156 and a well cavity 158 between open side 154 and closed face 156. According to some embodiments, as described above, one or more of at least one well 152 contains a cannabinoid concentrate 160 within its cavity 158. According to some embodiments, as described above, cannabinoid concentrate 160 comprises a cannabinoid selected from THC, CBD or both.

According to some embodiments, aerosol generating device 300 further comprises at least one translation mechanism 335. According to some embodiments (not shown), translation mechanism 335 comprises: a motor; and optionally a screw secured to the motor and configured to rotate responsive to the rotation of the motor. Alternatively, or additionally, according to some embodiments translation mechanism 335 comprises at least one rail. According to some embodiments, at least one heater 164 is secured to at least one translation mechanism 335.

According to some embodiments, at least one heater 164 is in electrical communication with at least one solenoid. According to some embodiments, aerosol generating device 300 further comprises a power source 350 configured to supply power to: at least one heater 164 and/or solenoid; processing unit 204; and rotatable tray actuator 320.

According to some embodiments, as shown in FIG. 13B, rotatable tray actuator 320 comprises: a gear 321 comprising a plurality of teeth 322 radially arrayed thereabout; a motor 323; and an axle 324. A first end of axle 324 is secured to motor 323 and a second end of axle 324 is secured to gear 321. According to some embodiments, rotatable tray 310 comprises a plurality of teeth 311 radially arrayed thereabout, teeth 311 configured to mesh with teeth 322 of gear 321. According to some embodiments, as shown in FIG. 13C, rotatable tray 310 and gear 321 are arranged in a spur gear configuration, i.e. where the rotational axis of gear 321 is parallel to rotational axis 330 of rotatable tray 310. Alternatively, according to some embodiments, as shown in FIG. 13D, rotatable tray 310 and gear 321 are arranged in a bevel gear configuration, i.e. where the rotational axis of gear 321 intersects rotational axis 330 of rotatable tray 310.

According to some embodiments, open side 154 of each well 152 faces outlet 102. According to some embodiments, at least one heater 164 is juxtaposed with rotatable tray 310. According to some embodiments, as described above in relation to aerosol generating device 100, at least one heater 164 is juxtaposed with closed face 156 of at least one well 152.

According to some embodiments, aerosol generating device 300 further comprises a housing 104. According to some embodiments, housing 104 comprises a mouthpiece 106. According to some embodiments, as described above, mouthpiece 106 extends between outlet 102 and a proximal mouthpiece side 107. According to some embodiments, at least one heater 164, rotatable tray actuator 320 and processing unit 204 are positioned within housing 104.

According to some embodiments (similar to that shown in FIG. 7), as described above in relation to cassette 150^{b}, rotatable tray 310 is positioned within an insertable/removable cassette and the cassette is positioned within housing 104. According to some embodiments, the cassette is detachably attachable within housing 104. The term “detachably attachable”, as used herein, means secured in such a way that it can be detached. According to some embodiments, at least one heater 164, rotatable tray actuator 320 and processing unit 204 are positioned within housing 104, external to the removable cassette. According to some embodiments, as described above in relation to aerosol generating device 100, housing 104 comprises a controlling member 200^{b}. According to some embodiments, at least one heater 164, rotatable tray actuator 320 and processing unit 204 are fixed within controlling member 200^{b} and the cassette is detachable from controlling member 200^{b}. Thus, as described above in relation to aerosol generating device 100, according to some embodiments, aerosol generating device 300 comprises a durable portion and a consumable portion.

According to some embodiments, as described above in relation to aerosol generating device 100, housing 104 comprises an inlet slot 178 configured and dimensioned to allow the cassette to be inserted therethrough. According to some embodiments, the cassette is juxtaposed with inlet slot 178 when detachably attachable within housing 104.

According to some embodiments (not shown), as described above in relation to cassette 150^{b} of aerosol generating device 100, the cassette comprises an identifier at an external surface thereof and aerosol generating device 300 further comprises a reader configured to identify the identifier. According to some embodiments, the reader is positioned within housing 104. According to some embodiments, the reader is positioned within controlling member 200^{b}. According to some embodiments, as described above, the reader is further configured to send identification signals indicative of the identification of the identifier to processing unit 204.

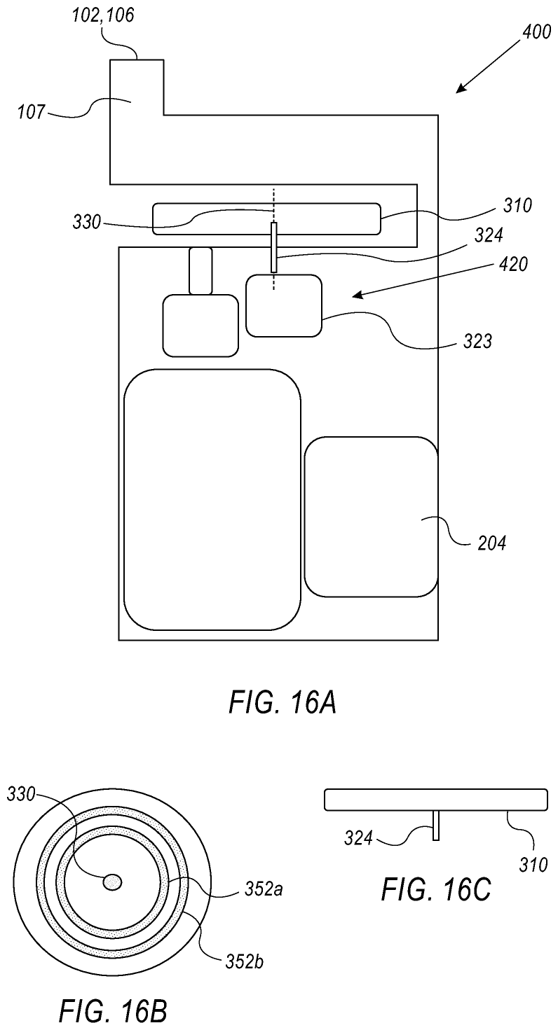

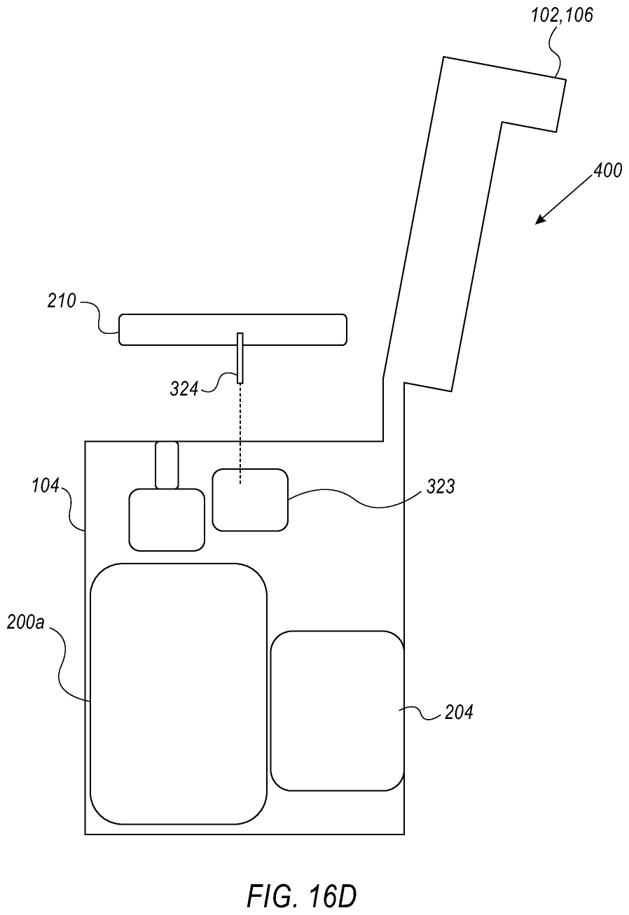

According to some embodiments, mouthpiece 106 is secured to housing 104. According to some embodiments, mouthpiece 106 is hingeably secured to housing 104, as described below, and shown, in relation to FIG. 16D. The term “hingeably secured”, as used herein, means secured via a hinge. The connection to the hinge can be either direct or via another element.

According to some embodiments, mouthpiece 106 is detachably attachable to housing 104. According to some embodiments (not shown), as described above in relation to cartridge 150^{a} of aerosol generating device 100, aerosol generating device 300 further comprises a cartridge. According to some embodiments, the cartridge comprises: rotatable tray 310; mouthpiece 106; and outlet 102. According to some embodiments, the cartridge is detachably attachable to housing 104. According to some embodiments, the cartridge is detachably attachable to controlling member 200^{a}.

According to some embodiments, as described above, the cartridge is intended to be disposable and for use until the cannabinoid concentrate 160 contained therein is consumed, whereas controlling member 200^{a} is durable and after consumption of the cannabinoid concentrate 160 contained in a first cartridge, a second cartridge may be mounted/assembled on controlling member 200^{a} for a further sequence of aerosolizations.

According to some embodiments, as described above in relation to aerosol generating device 100, the cartridge comprises an identifier. According to some embodiments, as described above in relation to aerosol generative device 100, aerosol generating device 300 further comprises a reader configured to identify the identifier of the cartridge. According to some embodiments, the reader is secured to housing 104. According to some embodiments, the reader is further configured to send identification signals indicative of the identification of the identifier of the cartridge to processing unit 204.

According to some embodiments, as shown in FIG. 13B, rotatable tray 310 comprises a plurality of wells 152. Although wells 152 are shown in FIG. 13B as comprising circular open sides 154, this is not meant to be limiting in any way and wells 152 can be provided in a variety of shapes, as described above.

According to some embodiments, wells 152 are radially arrayed about rotational axis 330. The term “radially arrayed”, as used herein, means positioned in relation to rotational axis 330 such that wells 152 generally form points along a circumference of a circle (not shown) surrounding rotational axis. According to some embodiments, the distances between rotational axis 330 and each well 152 is substantially equal. At least some of the plurality of wells 152 contain therewithin a cannabinoid concentrate 160. According to some embodiments, as described above, each cannabinoid concentrate 160 within each one of well cavities 158 of plurality of wells 152 comprises a cannabinoid selected from tetrahydrocannabinol (THC), cannabidiol (CBD) or both.

According to some embodiments, each well 152 is thermally isolated from the other wells 152. According to some embodiments, rotatable tray 310 is constructed with thermally isolating material, as described above in relation to tray 151. According to some embodiments, rotatable tray 310 comprises one or more thermally insulating materials. According to some embodiments, rotatable tray 310 is essentially composed of one or more thermally insulating materials. As used herein, the phrase “rotatable tray 310 is essentially composed of one or more thermally insulating materials” is intended to mean that aside from the well(s) (i.e. the closed face(s) 156 and side walls thereof) the rotatable tray 310 is made of thermally insulating material(s)

According to some embodiments, as described above in relation to tray 151, rotatable tray 310 comprises plurality of wells 152 and a plurality of inter-well joints 166. According to some embodiments, each one of inter-well joints 166 is inter-connecting between two adjacent wells 152. According to some embodiments, each one of inter-well joints 166 is made of a thermally insulating material. According to some embodiments, closed face 156 of each well 152 is made of thermally conductive material. According to some embodiments, the wall(s) of each well 152 is made of thermally conductive material.

According to some embodiments, as shown in FIG. 13E, the plurality of wells 152 comprises a first set of wells 152¹ and a second set of wells 152². According to some embodiments, first set of wells 152¹ comprises a respective plurality of wells 152¹ and second set of wells 152² comprises a respective plurality of wells 152². According to some embodiments, first set of wells 152¹ are radially arrayed about rotational axis 330 and second set of wells 152² are radially arrayed about first set of wells 152¹. Particularly, in such embodiments, second set of wells 152² are radially arrayed about rotational axis 330, yet further away than first set of wells 152¹. As a result, second set of wells 152² are radially arrayed about first set of wells 152¹. According to some embodiments, the distances between wells 152² and rotational axis 330 are greater than the distances between wells 152¹ and rotational axis 330.

According to some embodiments, each well 152¹ contains a first type of cannabinoid concentrate within its respective cavity 158 and each well 152² contains a second type of cannabinoid concentrate within its respective cavity. For example, wells 152¹ contain THC and wells 152² contain CBD, or vice versa.

Although two sets of wells 152 are shown, i.e. wells 152¹ and 152², this is not meant to be limiting in any way and any number of sets of wells 152 can be provided, each set of wells being radially arrayed about rotational axis 330.

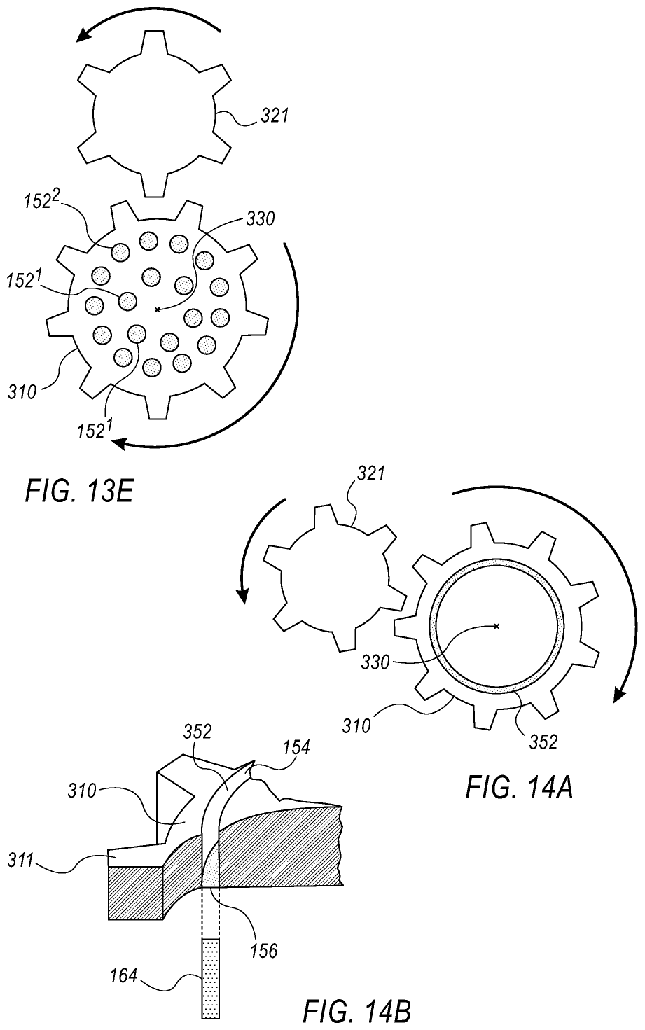

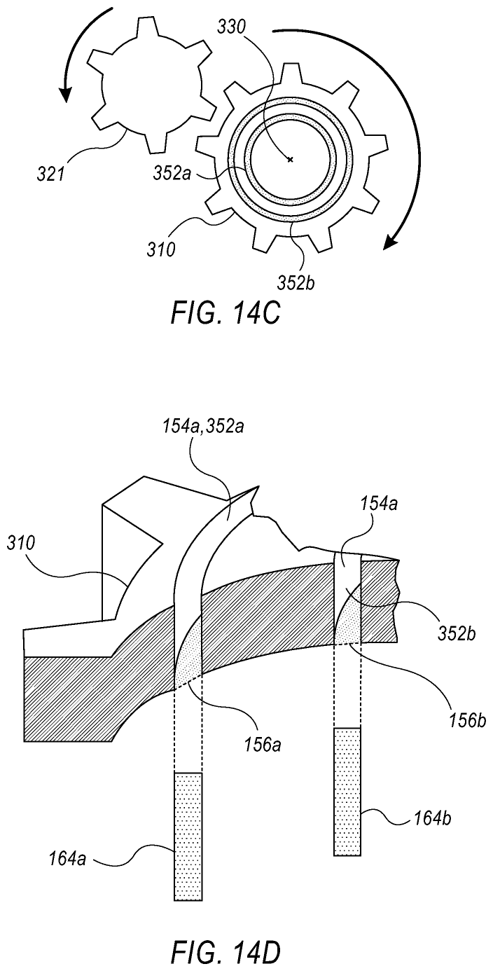

FIGS. 14A-14D constitute various views of portions of additional embodiments of aerosol generating device 300. Particularly, FIG. 14A constitutes a top view of various portions of a third embodiment of aerosol generating device 300. FIG. 14B constitutes a perspective view of a portion of the third embodiment of aerosol generating device 300. FIG. 14C constitutes a top view of various portions of a fourth embodiment of aerosol generating device 300. FIG. 14D constitutes a perspective view of a portion of the fourth embodiment of aerosol generating device 300.

According to some embodiments, as shown in FIGS. 14A-14B, rotatable tray 310 comprises a well 352. According to some embodiments, well 352 exhibits a shape radially extending about rotational axis 330. The term “radially extending”, as used herein, means that the respective well 352 surrounds rotational axis 330. Although FIG. 14A shows well 352 as completely surrounding rotational axis 330, this is not meant to be limiting in any way and well 352 can mostly surround rotational axis 330. According to some embodiments, well 352 is groove shaped, i.e. well 352 narrowly extends about rotational axis 330. According to some embodiments, well 352 is ring shaped with rotational axis 330 defining the center thereof.

As described above in relation to wells 152, according to some embodiments well 352 comprises an open side 354, a closed face 356 and a well cavity 358 defined between open side 354 and closed face 356. According to some embodiments, well 352 is in all respects similar to wells 152 described above, with the exception of the shape thereof. Well 352 contains a cannabinoid concentrate 160 in well cavity 358, as described above in relation to well cavities 158.

According to some embodiments, as shown in FIGS. 14C-14D, rotatable tray 310 comprises a first well 352a and a second well 352b, each of wells 352a and 352b exhibiting a respective shape radially extending about rotational axis 330. According to some embodiments, second well 352b radially extends about well 352a. As described above, although first well 352a and second well 352b are shown as completely surrounding rotational axis 330, this is not meant to be limiting in any way. According to other embodiments, well 352a and/or well 352b can mostly surround rotational axis 330. Although two wells 352 are illustrated, i.e. wells 352a and 352b, this is not meant to be limiting in any way, and any number of wells 352 can be provided.

As described above in relation to sets of wells 152¹ and 152², according to some embodiments well 352a contains a first type of cannabinoid concentrate within its respective cavity 158 and well 352b contains a second type of cannabinoid concentrate within its respective cavity. According to some embodiments, as described above, well 352a is thermally isolated from well 352b.

According to some embodiments, as described above in relation to well cavities 158 of wells 152, each cannabinoid concentrate 160 within each one of well cavities 358 comprises at least 10% cannabinoids w/w. According to some embodiments, each cannabinoid concentrate 160 within each one of well cavities 358 comprises at least 15% cannabinoids w/w. According to some embodiments, each cannabinoid concentrate 160 within each one of well cavities 358 comprises at least 20% cannabinoids w/w. According to some embodiments, each cannabinoid concentrate 160 within each one of well cavities 358 comprises at least 25% cannabinoids w/w. According to some embodiments, each cannabinoid concentrate 160 within each one of well cavities 358 comprises at least 30% cannabinoids w/w. According to some embodiments, each cannabinoid concentrate 160 within each one of well cavities 358 comprises at least 40% cannabinoids w/w. According to some embodiments, each cannabinoid concentrate 160 within each one of well cavities 358 comprises at least 50% cannabinoids w/w. According to some embodiments, each cannabinoid concentrate 160 within each one of well cavities 358 comprises at least 60% cannabinoids w/w. According to some embodiments, each cannabinoid concentrate 160 within each one of well cavities 358 comprises at least 70% cannabinoids w/w.

According to some embodiments, each cannabinoid concentrate 160 within each one of well cavities 358 comprises at least 10% THC w/w. According to some embodiments, each cannabinoid concentrate 160 within each one of well cavities 358 comprises at least 15% THC w/w. According to some embodiments, each cannabinoid concentrate 160 within each one of well cavities 358 comprises at least 20% THC w/w. According to some embodiments, each cannabinoid concentrate 160 within each one of well cavities 358 comprises at least 25% THC w/w. According to some embodiments, each cannabinoid concentrate 160 within each one of well cavities 358 comprises at least 30% THC w/w. According to some embodiments, each cannabinoid concentrate 160 within each one of well cavities 358 comprises at least 40% THC w/w. According to some embodiments, each cannabinoid concentrate 160 within each one of well cavities 358 comprises at least 50% THC w/w. According to some embodiments, each cannabinoid concentrate 160 within each one of well cavities 358 comprises at least 60% THC w/w. According to some embodiments, each cannabinoid concentrate 160 within each one of well cavities 358 comprises at least 70% THC w/w.

According to some embodiments, each cannabinoid concentrate 160 within each one of well cavities 358 comprises at least 10% CBD w/w. According to some embodiments, each cannabinoid concentrate 160 within each one of well cavities 358 comprises at least 15% CBD w/w. According to some embodiments, each cannabinoid concentrate 160 within each one of well cavities 358 comprises at least 20% CBD w/w. According to some embodiments, each cannabinoid concentrate 160 within each one of well cavities 358 comprises at least 25% CBD w/w. According to some embodiments, each cannabinoid concentrate 160 within each one of well cavities 358 comprises at least 30% CBD w/w. According to some embodiments, each cannabinoid concentrate 160 within each one of well cavities 358 comprises at least 40% CBD w/w. According to some embodiments, each cannabinoid concentrate 160 within each one of well cavities 358 comprises at least 50% CBD w/w. According to some embodiments, each cannabinoid concentrate 160 within each one of well cavities 358 comprises at least 60% CBD w/w. According to some embodiments, each cannabinoid concentrate 160 within each one of well cavities 358 comprises at least 70% CBD w/w.

It is to be understood that the phrase—cannabinoid concentrate 160 within each one of well cavities 358 comprises at least X % of a specified material w/w—mean that value of the weight of the specified material divided by the weight of the cannabinoid concentrate 160 is at least X %.

According to some embodiments, the combined weight of cannabinoids within rotatable tray 310 is at least 10% w/w compared to the combined weight of cannabinoid concentrate 160 within all the well cavities 158 and/or 358. According to some embodiments, the combined weight of cannabinoids within rotatable tray 310 is at least 20% w/w compared to the combined weight of cannabinoid concentrate 160 within all the well cavities 158 and/or 358. According to some embodiments, the combined weight of cannabinoids within rotatable tray 310 is at least 30% w/w compared to the combined weight of cannabinoid concentrate 160 within all the well cavities 158 and/or 358. According to some embodiments, the combined weight of cannabinoids within rotatable tray 310 is at least 40% w/w compared to the combined weight of cannabinoid concentrate 160 within all the well cavities 158 and/or 358. According to some embodiments, the combined weight of cannabinoids within rotatable tray 310 is at least 50% w/w compared to the combined weight of cannabinoid concentrate 160 within all the well cavities 158 and/or 358. According to some embodiments, the combined weight of cannabinoids within rotatable tray 310 is at least 60% w/w compared to the combined weight of cannabinoid concentrate 160 within all the well cavities 158 and/or 358. According to some embodiments, the combined weight of cannabinoids within rotatable tray 310 is at least 70% w/w compared to the combined weight of cannabinoid concentrate 160 within all the well cavities 158 and/or 358.

According to some embodiments, the combined weight of THC within rotatable tray 310 is at least 10% w/w compared to the combined weight of cannabinoid concentrate 160 within all the well cavities 158 and/or. According to some embodiments, the combined weight of THC within rotatable tray 310 is at least 20% w/w compared to the combined weight of cannabinoid concentrate 160 within all the well cavities 158 and/or 358. According to some embodiments, the combined weight of THC within rotatable tray 310 is at least 30% w/w compared to the combined weight of cannabinoid concentrate 160 within all the well cavities 158 and/or 358. According to some embodiments, the combined weight of THC within rotatable tray 310 is at least 40% w/w compared to the combined weight of cannabinoid concentrate 160 within all the well cavities 158 and/or 358. According to some embodiments, the combined weight of THC within rotatable tray 310 is at least 50% w/w compared to the combined weight of cannabinoid concentrate 160 within all the well cavities 158 and/or 358. According to some embodiments, the combined weight of THC within rotatable tray 310 is at least 60% w/w compared to the combined weight of cannabinoid concentrate 160 within all the well cavities 158 and/or 358. According to some embodiments, the combined weight of THC within rotatable tray 310 is at least 70% w/w compared to the combined weight of cannabinoid concentrate 160 within all the well cavities 158 and/or 358.

According to some embodiments, the combined weight of CBD within rotatable tray 310 is at least 10% w/w compared to the combined weight of cannabinoid concentrate 160 within all the well cavities 158 and/or 358. According to some embodiments, the combined weight of CBD within rotatable tray 310 is at least 20% w/w compared to the combined weight of cannabinoid concentrate 160 within all the well cavities 158 and/or 358. According to some embodiments, the combined weight of CBD within rotatable tray 310 is at least 30% w/w compared to the combined weight of cannabinoid concentrate 160 within all the well cavities 158 and/or 358. According to some embodiments, the combined weight of CBD within rotatable tray 310 is at least 40% w/w compared to the combined weight of cannabinoid concentrate 160 within all the well cavities 158 and/or 358. According to some embodiments, the combined weight of CBD within rotatable tray 310 is at least 50% w/w compared to the combined weight of cannabinoid concentrate 160 within all the well cavities 158 and/or 358. According to some embodiments, the combined weight of CBD within rotatable tray 310 is at least 60% w/w compared to the combined weight of cannabinoid concentrate 160 within all the well cavities 158 and/or 358. According to some embodiments, the combined weight of CBD within rotatable tray 310 is at least 70% w/w compared to the combined weight of cannabinoid concentrate 160 within all the well cavities 158 and/or 358.

According to some embodiments, as further shown in FIG. 14D, aerosol generating device 300 comprises a pair of heaters 164. According to some embodiments, a first heater 164a is juxtaposed with first well 352a and a second heater 164b is juxtaposed with second well 352b. Thus, in such embodiments, a distance between second heater 164b and rotational axis 330 is greater than a distance between first heater 164a and rotational axis 330. As specified above, although two wells 352 are illustrated, i.e. wells 352a and 352b, this is not meant to be limiting in any way, and any number of wells 352 can be provided. Similarly, although two heaters (164a and 164b) are illustrated this is not meant to be limiting in any way, and any number of heaters 164 can be provided. It is, however, to be understood that in such configuration, the number of heaters 164 preferably matches the number of wells 352.

According to some embodiments, processing unit 204 is configured to operate rotatable tray actuator 320 and at least one heater 164. Particularly, processing unit 204 controls rotatable tray actuator 320 to rotate rotatable tray 310 about rotation axis 330. In an embodiment where rotatable tray actuator 320 comprises gear 321 and a motor 322, processing unit 204 operates motor 322 to rotate gear 321. The rotation of gear 321 causes rotatable tray 310 to rotate about rotation axis 330 due to the mesh of teeth 322 of gear 321 with teeth 311 of rotatable tray 311. According to some embodiments, processing unit 204 operates rotatable tray actuator 320 to rotate rotatable tray 310 about rotation axis 330 by a predetermined rotation angle. According to some embodiments, where a plurality of wells 152 are provided, radially arrayed about rotation axis 330, the predetermined rotation angle is equal to 360 degrees divided by the number of wells 152.

According to some embodiments, processing unit 204 is configured to operate at least one heater 164 to generate heat at a predetermined temperature. According to some embodiments, processing unit 204 is configured to operate at least one heater 164 to elevate the temperature of the at least one well 152 to a predetermined temperature. According to some embodiments, each time processing unit 204 operates rotatable tray actuator 320 to rotate rotatable tray 310 about rotation axis 330, the respective heater 164 faces a respective well 152 and optionally is contact therewith. Thus, when the respective heater 164 is operated the temperature of the respective well 152 is elevated.

According to some embodiments, where a first heater 164a and a second heater 164b are provided, and where a first set of wells 152¹ and a second set of wells 152² are provided, as described above, after the rotation of rotatable tray 310 first heater 164a is juxtaposed with a respective well 152¹ and second heater 164b is juxtaposed with a respective well 152². Thus, when heaters 164a and 164b are operated, the temperature of each of the respective well 152¹ and well 152² is elevated.

According to some embodiments, where a well 352 is provided, at least one heater 164 elevates the temperature of at least a portion of well 352. According to some embodiments, a heater 164 is juxtaposed with well 352 and heater 164 elevates the temperature of the portion of well 352 juxtaposed therewith. According to some embodiments, where a first well 352a and a second well 352b are provided, first heater 164a is juxtaposed with first well 352a and second heater 164b is juxtaposed with second well 352b, as shown in FIG. 14D. In such an embodiment, first heater 164a elevates the temperature of at least a portion of first well 352a and second heater 164b elevates the temperature of at least a portion of second well 352b. According to some embodiments, first heater 164a elevates the temperature of the portion of first well 352a juxtaposed therewith and second heater 164b elevates the temperature of the portion of second well 352b juxtaposed therewith.

According to some embodiments, where at least one heater 164 is in electrical communication with at least one solenoid, processing unit 204 controls power source 350 to generate an electric current that flows through at least one solenoid, which then provides power to at least one heater 164.

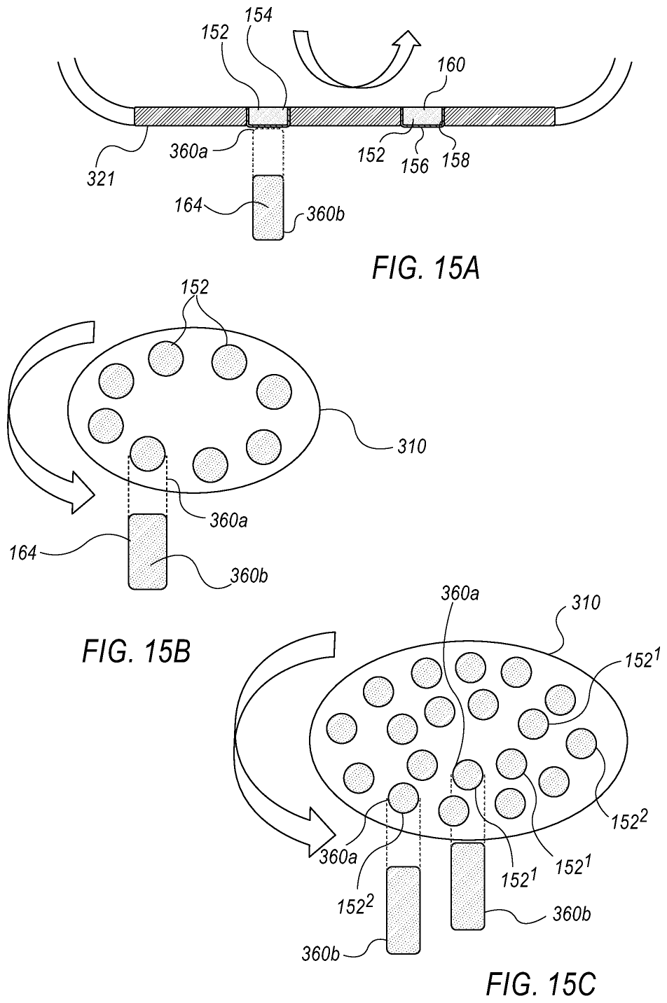

According to some embodiments, where aerosol generating device 300 further comprises at least one translation mechanism 335, processing unit 204 operates at least one translation mechanism 335 to translate at least one heater 164 between a first position 360a and a second position 360b in relation to rotatable tray 310, as shown in FIGS. 15A-15C.

Particularly, FIG. 15A constitutes a cross-sectional view of a portion of rotatable tray 310 and a heater 164, FIG. 15B constitutes a conceptual illustration of the positions of a heater 164 in relation to a plurality of wells 152, and FIG. 15C constitutes a conceptual illustration of the positions of a first heater 164a and a second heater 164b in relation to a first set of wells 152¹ and a second set of wells 152².

According to some embodiments, a distance between first position 360a and rotatable tray 310 is less than a distance between second position 360b and rotatable tray 310. Particularly, according to some embodiments, the distance between a heater 164 and a respective well 152 when in first position 360a is less than the distance between the heater 164 and the respective well 152 when in second position 360b. Similarly, according to some embodiments, the distance between a heater 164 and a respective well 352 when in first position 360a is less than the distance between the heater 164 and the respective well 352 when in second position 360b. Similarly, according to some embodiments, the distance between a first heater 164a and a respective well 152¹ when in first position 360a is less than the distance between the first heater 164a and the respective well 152¹ when in second position 360b. Similarly, according to some embodiments, the distance between a second heater 164b and a respective well 152² when in first position 360a is less than the distance between the second heater 164b and the respective well 152² when in second position 360b. Similarly, according to some embodiments, the distance between first heater 164a and well 352a when in first position 360a is less than the distance between first heater 164a and well 352a when in second position 360b. Similarly, according to some embodiments, the distance between second heater 164b and well 352b when in first position 360a is less than the distance between second heater 164b and well 352b when in second position 360b.

According to some embodiments, in first position 360a at least one heater 164 is in contact with rotatable tray 310. Thus, according to some embodiments, when operating at least one heater 164, processing unit 204 operates at least one translation mechanism 335 to translate at least one heater 164 to first position 360a such that the heat generated thereby elevates the temperature of the respective well 152 and/or 352.

Similarly, according to some embodiments, when ceasing operation of at least one heater 164, processing unit 204 operates at least one translation mechanism 335 to translate at least one heater 164 to second position 360b. Advantageously, after being translated to the second position residual heat generated by at least one heater 164 does not reach rotatable tray 310. Further advantageously, after being translated to the second position at least one heater 164 will not interfere with the rotation of rotatable tray 310.

As a result of the elevated temperature of the respective well 152 and/or 352, the respective cannabinoid concentrate 160, the respective cannabinoid concentrate 160 is vaporized, according to some embodiments, the vaporized cannabinoid concentrate 160 exiting through outlet 102, as described above in relation to aerosol generating device 100.

According to some embodiments, as described above, the predetermined temperature is in the range of 160° C. to 480° C.

According to some embodiments, the predetermined temperature is at least 160° C. According to some embodiments, the predetermined temperature is at least 180° C. According to some embodiments, the predetermined temperature is at least 200° C. According to some embodiments, the predetermined temperature is at least 250° C. According to some embodiments, the predetermined temperature is at least 300° C.

According to some embodiments, the predetermined temperature is no more than 480° C. According to some embodiments, the predetermined temperature is no more than 450° C. According to some embodiments, the predetermined temperature is no more than 425° C. According to some embodiments, the predetermined temperature is no more than 400° C.

FIGS. 16A-16D constitute various views of portions of an aerosol generating device 400, according to some embodiments. Particularly, FIG. 16A constitutes a cross-sectional view of aerosol generating device 400 in a closed configuration. FIGS. 16B-16C constitute a top view and a side view, respectively, of a portion of aerosol generating device 400. FIG. 16D constitutes a cross-sectional view of aerosol generating device 400 in an open configuration.

Aerosol generating device 400 is in all respects similar to aerosol generating device 300, with the exception that rotatable tray actuator 320 is replaced with rotatable tray actuator 420. Rotatable tray actuator 420 comprises: a motor 323; and an axle 324. A first end of axle 324 is secured to motor 323 and a second end of axle 324 is secured to rotatable tray 310. According to some embodiments, axle 324 extends along rotation axis 330. As shown in FIGS. 16B-16C, according to some embodiments, rotatable tray 310 does not include a gear.

According to some embodiments, processing unit 204 operates motor 323 to rotate axle 324, thereby rotating rotatable tray 310 about rotation axis 330. As described above, according to some embodiments, processing unit 204 further operates at least one heater 164 to heat at least one well 152 and/or 352. Although FIG. 16B is shown in an embodiment where rotatable tray 310 comprises a first well 352a and a second well 352b, this is not meant to be limiting in any way. According to some embodiments, as described above in relation to aerosol generating device 300, rotatable tray 310 comprises: a single well 352; a first well 352a and a second well 352b; a plurality of wells 152; and/or a first set of wells 152¹ and a second set of wells 152².

According to some embodiments, processing unit 204 is configured to operate rotatable tray actuator 420 and at least one heater 164. Particularly, processing unit 204 controls rotatable tray actuator 420 to rotate rotatable tray 310 about rotation axis 330 using axle 324. The rotation of axle 324 causes rotatable tray 310 to rotate about rotation axis 330 due to connection there between. According to some embodiments, processing unit 204 operates rotatable tray actuator 420 to rotate rotatable tray 310 about rotation axis 330 by a predetermined rotation angle. According to some embodiments, where a plurality of wells 152 are provided, radially arrayed about rotation axis 330, the predetermined rotation angle is equal to 360 degrees divided by the number of wells 152.

According to some embodiments, as shown in FIG. 16D, mouthpiece 106 is hingeably secured to controlling member 200a of housing 104. According to some embodiments, after hingeably opening mouthpiece 106 rotatable tray 310 can be removed from controlling member 200a. According to some embodiments, as described above, rotatable tray 310 is positioned within a removable cassette, such that the cassette can be removed after opening mouthpiece 106.

The above has been described and illustrated in relation to embodiments where rotatable tray 310 comprises at least one well 152 or at least one well 352, however this is not meant to be limiting in any way. According to some embodiments (not shown), rotatable tray 310 comprises one or more wells 152 and one or more wells 352. According to some embodiments, a respective heater 164 is provided for each wells 352 and for each well 152, or set of wells 152.

It is understood that aspect and embodiments described herein include “consisting” and/or “consisting essentially of” aspects and embodiments. As used herein, the singular form “a”, “an”, and “the” includes plural references unless indicated otherwise.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. An aerosol generating device comprising:
a rotatable tray comprising at least one well having an open side, a closed face and a cavity there between, wherein the at least one well contains a cannabinoid concentrate within its cavity;
a rotatable tray actuator configured to rotate the rotatable tray around a rotational axis;
at least one heater juxtaposed with the rotatable tray;
a processing unit configured to operate the rotatable tray actuator and the at least one heater; and
an outlet,
wherein the open side of the at least one well faces the outlet.

2. The aerosol generating device of claim 1, wherein the at least one heater is configured to elevate the temperature of the at least one well.

3. The aerosol generating device of claim 1, wherein the at least one well comprises a plurality of wells, the plurality of wells radially arrayed about the rotational axis.

4. The aerosol generating device of claim 3, wherein each of the plurality of wells is thermally isolated from the other wells.

5. The aerosol generating device of claim 3, or wherein the plurality of wells comprises a first set of wells and a second set of wells,
wherein the first set of wells are radially arrayed about the rotational axis and the second set of wells are radially arrayed about the first set of wells,
wherein each of the first set of wells contains a first type of cannabinoid concentrate within its cavity, and
wherein each of the second set of wells contains a second type of cannabinoid concentrate within its cavity.

6. (canceled)

7. The aerosol generating device of claim 5 or 6, wherein the at least one heater comprises a pair of heaters, a first of the pair of heaters juxtaposed with the first set of wells and the second pair of heaters juxtaposed with the second set of wells such that a distance between the rotational axis and the second heater is greater than a distance between the rotational axis and the first heater.

8. The aerosol generating device of claim 1, wherein the at least one well exhibits a shape radially extending about the rotational axis.

9. (canceled)

10. (canceled)

11. (canceled)

12. The aerosol generating device of claim 1, wherein the rotatable tray actuator comprises a gear comprising a plurality of teeth, and
wherein the rotatable tray comprises a plurality of teeth, the plurality of teeth of the rotatable tray configured to mesh with the plurality of teeth of the gear.

13. The aerosol generating device of claim 1, wherein the rotatable tray actuator comprises an axle secured to the rotatable tray and extending along the rotational axis.

14. The aerosol generating device of claim 1, further comprising at least one translation mechanism configured to translate the at least one heater between a first position and a second position in relation to the rotatable tray, a distance between the first position and the rotatable tray being less than a distance between the second position and the rotatable tray,
wherein the processing unit is further configured to:
operate the at least one translation mechanism to translate the at least one heater from the first position to the second position;
operate the rotatable tray actuator to rotate the rotatable tray about the rotation axis by a predetermined amount; and
subsequent to the rotation of the rotatable tray, operate the at least one translation mechanism to translate the at least one heater from the second position to the first position.

15. The aerosol generating device of claim 14, wherein in the first position the at least one heater is in contact with the rotatable tray.

16. (canceled)

17. The aerosol generating device of claim 1, further comprising:
a housing;
a cassette positioned within the housing;
an identifier positioned on a face of the cassette; and
a reader positioned within the housing,
wherein the rotatable tray is positioned within the cassette,
wherein the at least one heater, the rotatable tray actuator and the processing unit are positioned within the housing, external to the cassette, and
wherein the reader is configured to:
identify the identifier; and
output a signal indicative of an identification of the identifier.

18. The aerosol generating device of claim 1, further comprising:
a cassette, the rotatable tray positioned within the cassette; and
a housing,
wherein the cassette is detachably attachable within the housing, and
wherein the at least one heater, the rotatable tray actuator and the processing unit are positioned within the housing, external to the cassette.

19. The aerosol generating device of claim 18, wherein the housing comprises an inlet slot configured and dimensioned to allow the cassette to be inserted therethrough, the cassette juxtaposed with the inlet slot when detachably attachable within the housing.

20. The aerosol generating device of claim 18, wherein the housing comprises a mouthpiece extending from the outlet, and
wherein the mouthpiece is hingeably or detachably attachable to the housing.

21. The aerosol generating device of claim 1, further comprising:
a housing;
a cartridge detachably coupled to the housing;
an identifier positioned on the cartridge; and
a reader secured to the housing,
wherein the rotatable tray is positioned within the cartridge,
wherein the at least one heater, the rotatable tray actuator and the processing unit are positioned within the housing, external to the cartridge,
wherein the housing comprises a mouthpiece extending from the outlet, and
wherein the reader is configured to:
identify the identifier; and
output a signal indicative of an identification of the identifier.

22. The aerosol generating device of claim 1, wherein the at least one heater comprises at least one induction coil or at least one laser.

23. (canceled)

24. An aerosol generating device comprising:
a tray comprising a plurality of wells, each having an open side, a closed face and a cavity there between, wherein at least some of the wells contain a cannabinoid concentrate within their cavity;
a plurality of heaters, each heater is configured to elevate the temperature of one respective well of the plurality of wells;
a processing unit configured to separately operate each heater, thereby to elevate the temperature within each well individually; and
an outlet,
wherein the closed face of each well is facing the processing unit and the open side of each well is facing the outlet.

25. The aerosol generating device of claim 24, wherein each heater is in contact with the closed face of the well heated thereby.

26. (canceled)

27. (canceled)

28. (canceled)

29. (canceled)

30. (canceled)

31. The aerosol generating device of claim 24, comprising:
a housing, which houses the processing unit, a mouthpiece comprising the outlet and a power source compartment; and
a cassette comprising the tray and the plurality of heaters;
wherein the housing comprises an inlet slot configured for insertion of the cassette into the housing, wherein upon insertion of cassette into the inlet slot, the aerosol generating device is assembled, wherein upon assembly, the processing unit forms an electric contact with each one of the heaters separately.

32. (canceled)
